(19) United States
(12) Patent Application Publication
(10) Pub. No.: US 2013/0295570 A1
(43) Pub. Date: Nov. 7, 2013

(54) COMPOSITIONS AND METHODS FOR NUCLEIC ACID BASED DIAGNOSTIC ASSAYS

(71) Applicants: Lawrence J. Wangh, Auburndale, MA (US); J. Aquiles Sanchez, Framingham, MA (US)

(72) Inventors: Lawrence J. Wangh, Auburndale, MA (US); J. Aquiles Sanchez, Framingham, MA (US)

(73) Assignee: BRANDEIS UNIVERSITY, Waltham, MA (US)

(21) Appl. No.: 13/878,044

(22) PCT Filed: Oct. 7, 2011
(86) PCT No.: PCT/US11/55237

§ 371 Date: Jul. 1, 2013

Related U.S. Application Data
(60) Provisional application No. 61/390,760, filed on Oct. 7, 2010.

(51) Int. Cl. C12Q 1/68

(52) U.S. Cl. CPC C12Q 1/6883
USPC 435/6.11; 536/24.3; 702/19

(57) ABSTRACT

The present invention relates to compositions and methods for nucleic acid based diagnostic assays. In particular, the present invention provides probes and non-amplifiable controls for asymmetric PCR and other amplification modalities. In some embodiments, the present invention provides probe design criteria for probes for use in amplification/detection assays. Further embodiments of the present invention provide non-amplifiable controls for use in generating reference probe signal ratios in amplification detection assays.

### CROSS-REFERENCE TO RELATED APPLICATIONS

The present application claims priority to U.S. Provisional Patent Application Ser. No. 61/390,760, filed Oct. 7, 2010, which is incorporated by reference in its entirety.

### FIELD OF THE INVENTION

The present invention relates to compositions and methods for nucleic acid based diagnostic assays. In particular, the present invention provides probes and non-amplifiable controls targets for asymmetric PCR and other amplification modalities. In some embodiments, the present invention provides probe design criteria for probes for use in amplification/detection assays. Further embodiments of the present invention provide non-amplifiable control targets that are added to an amplification detection assay prior to amplification for use in generating reference probe signals or reference probe signal ratios.

### BACKGROUND

As the volume of genetic sequence information available increases, genomics research and subsequent drug design efforts increase as well. A number of institutions are actively mining the available genetic sequence information to identify correlations between genes, gene expression and phenotypes (e.g., presence and/or identity of pathogens in sample, disease states, metabolic responses, and the like).

Despite substantial efforts made, existing approaches for analyzing nucleic acid molecules still suffer from inaccuracies and/or inefficiencies and may not provide sufficient information that is accurate, fast, and cost effective.

Thus, the art is in need of improved methodologies.

### SUMMARY OF THE INVENTION

The present invention relates to compositions and methods for nucleic acid based diagnostic assays. In particular, the present invention provides probes and non-amplifiable control targets for asymmetric PCR and other amplification modalities. In some embodiments, the present invention provides probe design criteria for probes for use in amplification/detection assays. Further embodiments of the present invention provide non-amplifiable control targets that are added to an amplification detection assay prior to amplification for use in generating reference probe signals or reference probe signal ratios.

In some embodiments, the present invention provides a method for designing a mismatch tolerant probe, comprising: a) selecting a candidate probe sequence that is perfectly complementary to one allele of a polymorphic target sequence (e.g. a single nucleotide polymorphism, SNP), the probe sequence having 5′ and 3′ ends; and b) designing a probe from said candidate probe sequence by introducing one or more of the following modifications to said sequence: i) addition of a nucleotide “tail” at the 5′ and/or 3′ ends of the candidate probe sequence of at least 2 nucleotides that is not complementary to the target sequence; ii) addition of a mismatch nucleotide at least one nucleotide away from the position of the polymorphic allele on the target sequence; iii) positioning the portion of the candidate probe sequence that hybridizes to the polymorphic sequence on the target at least 2 nucleotides away from either the 5′ or the 3′ end of the probe. In some embodiments, the modified sequence is labeled for use as a probe (e.g., by placing a fluorophore at an end of the probe sequence and a quencher at the other end). In some embodiments, the modified probe hybridizes to, a) at least 90% (e.g., 95.5% or 100%) of the target sequences that are complementary with the probe sequence at the site of the polymorphic allele and to less than 10% (e.g., 3% or 0%) of the target sequences that are mismatched with the probe sequence at the site of the polymorphic allele at a detection temperature and, b) to at least 90% (e.g., 100%) of all target allelic variants at a lower detection temperature. In some embodiments, the modified probe is a probe for use in asymmetric PCR detection assays (e.g., LATE-PCR). In some embodiments related to asymmetric PCR assays, the detection temperature where the probe exhibits maximum discrimination for binding to the target complementary at the site of the polymorphic allele is at least 8° C.-10° C. below the melting temperature of the amplification primer at the lowest concentration. Further embodiments of the present invention provide a modified probe produced by the above-described method.

Additional embodiments of the present invention provide a non-amplifiable control target that is added prior to the start of an asymmetric PCR reaction comprising a non-amplifiable oligonucleotide that, a) is not complementary to the primers used in PCR, and b) corresponds to binding site of a mismatch-tolerant nucleic acid probe on the target sequence to be amplified. In some embodiments, the non-amplifiable oligonucleotide target is blocked at its 3′ ends. In some embodiments, the non-amplifiable oligonucleotide target comprises at least 6 nucleotides flanking each side of the mismatch-tolerant probe first binding site. In some embodiments, the non-amplifiable oligonucleotide is at a defined concentration of at least 50 nM.

Embodiments of the present invention provide a method comprising: a) contacting a mismatch tolerant probe with a non-amplifiable control target that is added prior to the start of amplification, wherein the non-amplifiable control target comprises a sequence that: i) is not complementary to the primers used in PCR; and ii) corresponds to a binding site of a mismatch-tolerant nucleic acid probe on the target sequence to be amplified; b) generating a reference fluorescence signal value at one or more detection temperatures (e.g. to generate a melting curve) for the binding of said probe on said target prior to PCR; c) performing an asymmetric PCR that amplifies the intended target sequence; e) obtaining a post-PCR fluorescent signal at the same detection temperature used before PCR; f) subtracting the pre-PCR fluorescence signals from the post-PCR fluorescence signals at the corresponding temperatures to generate adjusted fluorescence signal values; and g) normalizing the adjusted fluorescent signal against the pre-PCR fluorescent signal to correct from variation in post-PCR fluorescent signals among replicate samples. In some embodiments, the non-amplifiable control oligonucleotide is present at a concentration of approximately 50 nM.

Additional embodiments of the present invention provide a non-amplifiable control target that is added prior to the start of an asymmetric PCR reaction, comprising: a) a first oligonucleotide target comprising a sequence corresponding to a first binding site of a mismatch-tolerant nucleic acid probe on a target to be amplifiable by PCR, wherein the sequence corresponds to a first allele of the first binding site and wherein the sequence comprises at least one nucleotide difference to a second allele of the binding site; and b) a second oligonucleotide target comprising a sequence corresponding to a second binding site of the mismatch-tolerant nucleic acid probe on said target to be amplified by PCR, wherein the sequence corresponds to a second allele of the first binding site and wherein the sequence comprises at least one nucleotide difference to the first allele of the first binding site. In some embodiments, the control oligonucleotide targets are not complementary to primers used in PCR and are therefore non-amplifiable by PCR. In some embodiments, the non-amplifiable oligonucleotide targets are blocked at their 3′ ends. In some embodiments, the non-amplifiable oligonucleotide targets comprise at least 6 nucleotides flanking each side of the mismatch-tolerant probe first and second binding sites. In some embodiments, the oligonucleotides are in equimolar amounts or at a predetermined molar ratio. In other embodiments, if both the first and the second oligonucleotide are present the concentration of the least abundant non-amplifiable oligonucleotide is at least 50 nM.

Embodiments of the present invention provide a method, comprising: a) contacting a mismatch tolerant probe with a non-amplifiable control target that is added prior to the start of amplification, wherein the non-amplifiable control target comprises: a) a first oligonucleotide target comprising a sequence corresponding to a first binding site of a mismatch-tolerant nucleic acid probe on the target to be amplifiable by PCR, wherein the sequence corresponds to a first allele of the first binding site, wherein the sequence comprises at least one nucleotide difference to a second allele of the binding site, and wherein the sequence is not complementary to the primers used for amplification; and b) a second oligonucleotide target comprising a sequence corresponding to a second binding site of the mismatch-tolerant nucleic acid probe on said target to be amplified by PCR, wherein the sequence corresponds to a second allele of the first binding site, wherein the sequence comprises at least one nucleotide difference to the first allele of the first binding site, and wherein the sequence is not complementary to the primers used for amplification; b) measuring fluorescence signals prior to PCR at at least three temperatures comprising a high temperature where the mismatch-tolerant probe does not bind to either of the allelic targets, a middle temperature wherein the mismatch tolerant probe binds to at least 90% (e.g., 100%) of the first allelic target and to at least 10% or less (e.g., 0%) of the second allelic target, and a third temperature where the mismatch-tolerant probe binds to at least 90% (e.g., 100%) of all first and second allelic targets; c) calculating a three-temperature fluorescent ratio (e.g., according to the formula (Fs-Ft)/(Fb-Ft), where Ft is the fluorescence at the first detection temperature, Fb is the fluorescence at the low temperature, and Fs is the fluorescence at the intermediate temperature; the fluorescent ratio defines a reference fluorescent ratio for the molar ratio of the first and second oligonucleotides before PCR); d) performing a asymmetric PCR that amplifies the polymorphic sequence; e) repeating step b) to obtain a post-PCR fluorescent signals at the same three detection temperatures; f) subtracting the pre-PCR fluorescence signals from the post-PCR fluorescence signals at the corresponding temperatures to generate adjusted fluorescence signal values, and g) repeating step c for said adjusted fluorescence signal values to generate post PCR fluorescent signal ratios. In some embodiments, similar pre-PCR fluorescence ratios and post PCR fluorescence ratios are indicative of a DNA sample with allelic ratios similar to the molar ratio of the first and second oligonucleotide controls. In some embodiments, pre-PCR fluorescence ratios different than post PCR fluorescence ratios are indicative of a DNA sample with allelic ratios different from the molar ratios of the first and second oligonucleotide controls. In some embodiments, the first and second oligonucleotides are present at a concentration of approximately 50 nM each.

Further embodiments of the present invention provide a method for performing asymmetric PCR (e.g., LATE PCR) using a probe comprising one or more mismatches to the target at a location distinct from the polymorphic target sequence (e.g., SNP). For example, in some embodiments the method comprises a) providing: i) a sample suspected of containing a nucleic acid sequence comprising a first and/or second variant of a polymorphic target sequence, ii) a labeled probe, wherein the probe comprises one or more mismatches to the nucleic acid sequence at a location other than at the polymorphic site in the target sequence (e.g., not precluding mismatches to some but not all alleles of the polymorphic target sequence), iii) a first variant temperature signal ratio, iv) a second variant temperature signal ratio, v) a temperature signal ratio indicative of the presence of both the first and the second variants, vi) a forward primer, and vii) a reverse primer; b) combining the sample, the labeled probe, the forward primer, and the reverse primer to generate a combined sample and treating the combined sample under amplification conditions such that: a first single-stranded amplicon is generated if the first variant is present, and a second single-stranded amplicon is generated if the second variant is present, wherein the first and second single-stranded amplicons each comprise the following identical sequences: i) a 5′ end corresponding to the sequence of the reverse primer, and iii) a 3′ end complementary to the forward primer; and wherein the first and second single-stranded amplicons do not have complete sequence identity; c) exposing the combined sample to multiple temperatures that allow the labeled probe to hybridize to the probe hybridization sequence and produce temperature-dependent signals; d) detecting the temperature-dependent signals at at least two temperatures; e) generating an experimental temperature signal ratio; and f) comparing the experimental temperature signal ratio with the first, second, and first/second variant temperature signal ratios, wherein a match between the experimental temperature signal ratio and the first or second variant temperature signal ratio identifies the presence of the first and/or second variant in the sample. In some embodiments, the forward primer comprises a limiting primer and the reverse primer comprises an excess primer, wherein the excess primer is added to the combined sample at a concentration at least five-times that of the limiting primer, and wherein the amplification conditions comprise asymmetric PCR conditions. In some embodiments, the asymmetric PCR conditions are LATE-PCR conditions, and wherein the initial melting temperature of the limiting primer is higher than or equal to the initial melting temperature of the excess primer. In some embodiments, the labeled probe comprises with one of the following modifications: i) addition of a nucleotide tail at the 5′ and/or 3′ ends of the probe sequence of at least two nucleotides (e.g., 2, 3, 4, 5, . . . ) that are not complementary to the polymorphic target sequence; ii) addition of a mismatch nucleotide at least one nucleotide away from the position of the polymorphic target sequence; and iii) positioning the portion of the probe that hybridizes to the polymorphic target sequence at least two nucleotides away from either the 5′ or the 3′ end of the probe. Additional embodiments are described herein.

### DESCRIPTION OF THE FIGURES

FIG. 1 shows normalized thermal profiles of a mismatch tolerant probe. In particular, FIG. 1 shows normalized thermal profiles of a mismatch-tolerant probe perfectly complementary to a SNP allele hybridized to targets that are either homozygous or heterozygous for said SNP allele (red and blue lines, respectively) or homozygous for the other SNP allele (green lines). The ratio of probe signals at middle and low temperatures normalized to the signals at the upper temperature reveals the percentage of targets with the interrogated SNP allele in the sample.

FIG. 2 shows the fluorescent ratio shift due to loss of heterozygosity (LOH). The fluorescent ratios shift up or down from the heterozygous values in accord with which allele is lost due to LOH.

FIG. 3 shows the VOMP melt profile criteria of exoR probes designed for LATE PCR LOH detection. Rightmost curve: Probe binding to matched allele target. Leftmost curve: Probe binding to mismatched allele target. Righmost vertical line: Temperature where the probe binds to at least 90% of matched target (actual 94%) and less than 5% of the mismatched target (actual 2.3%). Leftmost vertical line: Maximum temperature where the probe binds to 100% of the matched and mismatched targets.

FIG. 4 shows probes designed using the design criteria described in embodiments of the present invention.

FIG. 5 shows a schematic of LATE-PCR assays used in embodiments of the present invention.

FIG. 6 shows genotyping using LATE-PCR endpoint assays.

FIG. 7 shows use of non-amplifiable internal controls of embodiments of the present invention to generate reference fluorescent ratios for genotyping.

FIG. 8 shows the difference between the Pre-PCR fluorescent ratios and post-PCR fluorescent ratios for samples of different genotypes.

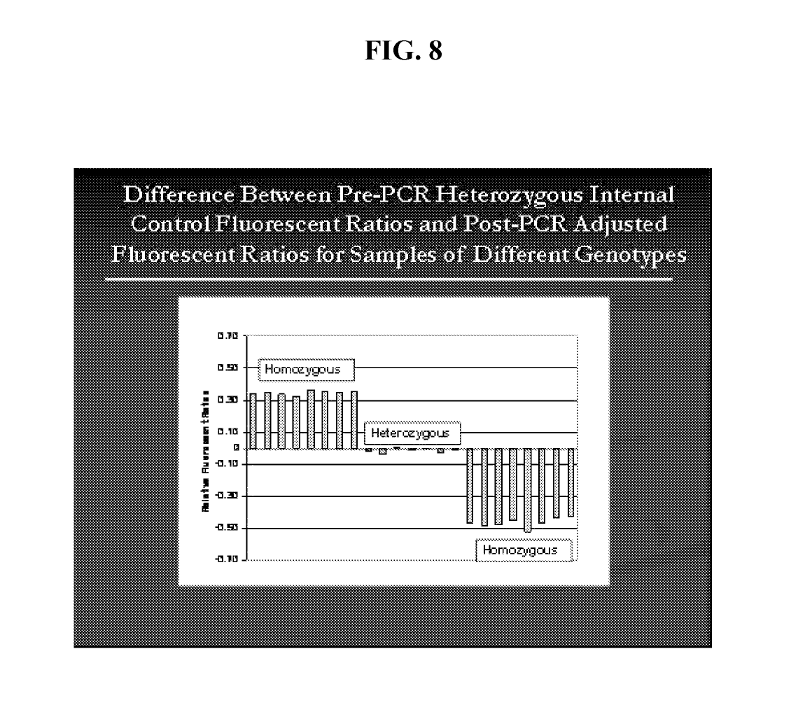

FIG. 9 shows the identification of three temperatures to be used for probe signal collection in LATE-PCR endpoint genotyping.

FIG. 10 shows the first derivative of the melting curve for pre-PCR fluorescent signal analysis.

FIG. 11 shows normalized fluorescent signals at the upper and lower temperatures PRE-PCR.

FIGS. 12A and 12B show the first derivative and normalized fluorescent ratios post PCR.

FIG. 13 shows the difference between pre-PCR rations and PCR products post PCR for different genotypes.

FIGS. 14A-C show exemplary probe designs.

### DEFINITIONS

As used herein, the phrase “probe hybridization sequence” is used is reference to a particular target sequence and a particular probe, and it is the sequence in the target sequence that hybridizes to the particular probe. The probe may be fully or partially complementary to the target sequence over the length of the probe hybridization sequence. In some embodiments, the probe hybridization sequence is labeled to enable its detection (e.g., with a fluorophore at one end and quencher at the other end).

As used herein, the term “amplicon” refers to a nucleic acid generated using primer pairs, such as those described herein. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable.

Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. In certain embodiments, the type of amplification is asymmetric PCR (e.g., LATE-PCR) which is described in, for example, U.S. Pat. No. 7,198,897, Sanchez et al., PNAS, 2004, 101(7):1933-1938, and Pierce et al., PNAS, 2005, 102(24):8609-8614, all of which are herein incorporated by reference in their entireties. In particular embodiments, LATE-PCR is employed using multiple end-point temperature detection (see, e.g., U.S. Pat. Pub. 2006/0177841 and Sanchez et al., BMC Biotechnology, 2006, 6:44, pages 1-14, both of which are herein incorporated by reference).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (T_{M}) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference. As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

In some embodiments, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N-6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N— isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more bioagents. Samples can include, for example, blood, saliva, urine, feces, anorectal swabs, vaginal swabs, cervical swabs, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. (Stryer et al., 1978, Ann. Rev. Biochem., 47:819; Selvin, 1995, Methods Enzymol., 246:300, each incorporated herein by reference). As used herein, the term “donor” refers to a fluorophore that absorbs at a first wavelength and emits at a second, longer wavelength. The term “acceptor” refers to a moiety such as a fluorophore, chromophore, or quencher that has an absorption spectrum that overlaps the donor's emission spectrum, and that is able to absorb some or most of the emitted energy from the donor when it is near the donor group (typically between 1-100 nm). If the acceptor is a fluorophore, it generally then re-emits at a third, still longer wavelength; if it is a chromophore or quencher, it then releases the energy absorbed from the donor without emitting a photon. In some embodiments, changes in detectable emission from a donor dye (e.g. when an acceptor moiety is near or distant) are detected. In some embodiments, changes in detectable emission from an acceptor dye are detected. In some embodiments, the emission spectrum of the acceptor dye is distinct from the emission spectrum of the donor dye such that emissions from the dyes can be differentiated (e.g., spectrally resolved) from each other.

Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral.

“T_{M},” or “melting temperature,” of an oligonucleotide describes the temperature (in degrees Celsius) at which 50% of the molecules in a population of a single-stranded oligonucleotide are hybridized to their complementary sequence and 50% of the molecules in the population are not-hybridized to said complementary sequence. The T_{M} of a primer or probe can be determined empirically by means of a melting curve. In some cases it can also be calculated. For the design of symmetric and asymmetric PCR primer pairs, balanced T_{M}'s are generally calculated by one of the three methods discussed earlier, that is, the “% GC”, or the “2(A+T) plus 4 (G+C)”, or “Nearest Neighbor” formula at some chosen set of conditions of monovalent salt concentration and primer concentration. In the case of Nearest Neighbor calculations the T_{M}'s of both primers will depend on the concentrations chosen for use in calculation or measurement, the difference between the T_{M}'s of the two primers will not change substantially as long as the primer concentrations are equimolar, as they normally are with respect to PCR primer measurements and calculations. T_{M [}1] describes the calculated T_{M} of a PCR primer at particular standard conditions of 1 micromolar (1 uM=10⁻⁶M) primer concentration, and 0.07 molar monovalent cations. In this application, unless otherwise stated, T_{M[}1] is calculated using Nearest Neighbor formula, T_{M}=ΔH/(ΔS+R ln(C/2))−273.15+12 log [M]. This formula is based on the published formula (Le Novere, N. (2001), “MELTING, Computing the Melting Temperature of Nucleic Acid Duplex,” Bioinformatics 17: 1226 7). ΔH is the enthalpy and ΔS is the entropy (both ΔH and ΔS calculations are based on Allawi and SantaLucia, 1997), C is the concentration of the oligonucleotide (10⁻⁶M), R is the universal gas constant, and [M] is the molar concentration of monovalent cations (0.07). According to this formula the nucleotide base composition of the oligonucleotide (contained in the terms ΔH and ΔS), the salt concentration, and the concentration of the oligonucleotide (contained in the term C) influence the T_{M}. In general for oligonucleotides of the same length, the T_{M} increases as the percentage of guanine and cytosine bases of the oligonucleotide increases, but the T_{M} decreases as the concentration of the oligonucleotide decreases. In the case of a primer with nucleotides other than A, T, C and G or with covalent modification, T_{M[}1] is measured empirically by hybridization melting analysis as known in the art.

“T_{M[}0]” means the T_{M} of a PCR primer or probe at the start of a PCR amplification taking into account its starting concentration, length, and composition. Unless otherwise stated, T_{M[}0] is the calculated T_{M} of a PCR primer at the actual starting concentration of that primer in the reaction mixture, under assumed standard conditions of 0.07 M monovalent cations and the presence of a vast excess concentration of a target oligonucleotide having a sequence complementary to that of the primer. In instances where a target sequence is not fully complementary to a primer it is important to consider not only the T_{M[}0] of the primer against its complements but also the concentration-adjusted melting point of the imperfect hybrid formed between the primer and the target. In this application, T_{M[}0] for a primer is calculated using the Nearest Neighbor formula and conditions stated in the previous paragraph, but using the actual starting micromolar concentration of the primer. In the case of a primer with nucleotides other than A, T, C and G or with covalent modification, T_{M[}0] is measured empirically by hybridization melting analysis as known in the art.

As used herein superscript X refers to the Excess Primer, superscript L refers to the Limiting Primer, superscript A refers to the amplicon, and superscript P refers to the probe.

T_{M}^{A} means the melting temperature of an amplicon, either a double-stranded amplicon or a single-stranded amplicon hybridized to its complement. In this application, unless otherwise stated, the melting point of an amplicon, or T_{M}^{A}, refers to the T_{M} calculated by the following % GC formula: T_{M}^{A}=81.5+0.41(% G+% C)−500/L+16.6 log [M]/(1+0.7 [M]), where L is the length in nucleotides and [M] is the molar concentration of monovalent cations.

T_{M[}0]^{P} refers to the concentration-adjusted melting temperature of the probe to its target, or the portion of probe that actually is complementary to the target sequence (e.g., the loop sequence of a molecular beacon probe). In the case of most linear probes, T_{M[}0]^{P} is calculated using the Nearest Neighbor formula given above, as for T_{M[}0], or preferably is measured empirically. In the case of molecular beacons, a rough estimate of T_{M[}0]^{P} can be calculated using commercially available computer programs that utilize the % GC method, see Marras, S. A. et al. (1999) “Multiplex Detection of Single-Nucleotide Variations Using Molecular Beacons,” Genet. Anal. 14:151 156, or using the Nearest Neighbor formula, or preferably is measured empirically. In the case of probes having non-conventional bases and for double-stranded probes, T_{M[}0]^{P} is determined empirically.

C_{T} means threshold cycle and signifies the cycle of a real-time PCR amplification assay in which signal from a reporter indicative of amplicons generation first becomes detectable above background. Because empirically measured background levels can be slightly variable, it is standard practice to measure the C_{T} at the point in the reaction when the signal reaches 10 standard deviations above the background level averaged over the 5-10 preceding thermal cycles.

As used herein, the term “non-amplifiable control” refers to non-amplifiable oligonucleotides targets for the detection probe that are added to a PCR sample to generate reference fluorescent ratios/signals. In some embodiments, these oligonucleotides targets lack complementary to the primers used for PCR amplification and are therefore non-amplifiable.

### DETAILED DESCRIPTION

The present invention relates to compositions and methods for nucleic acid based diagnostic assays. In particular, the present invention provides probes and non-amplifiable control targets for asymmetric PCR and other amplification modalities. In some embodiments, the present invention provides probe design criteria for probes for use in amplification/detection assays. Further embodiments of the present invention provide non-amplifiable control targets that are added to an amplification detection assay prior to amplification for use in generating reference probe signals or reference probe signal ratios.

The invention finds use in any application that identifies SNPs, other polymorphisms, or other sequences of interest. For example, embodiments of the present invention provide compositions and methods for use in screening and diagnostic assays that identify alleic imbalances due to chromosomal copy number variations (e.g. deletions, duplications), the presence of or identity of pathogenic nucleic acid in a sample, and the like. The invention also find used in reducing the scatter among fluorescent probe signals from replicate amplification reactions. Additional uses are within the scope of one of skill in the art.

### I. Asymmetric PCR

In some embodiments, the present invention provides probes and non-amplifiable control targets for use in amplification and detection assays. The methods described herein are not limited by the type of amplification that is employed. In certain embodiments, asymmetric PCR is employed, such as LATE-PCR.

PCR is a repeated series of steps of denaturation, or strand melting, to create single-stranded templates; primer annealing; and primer extension by a thermally stable DNA polymerase such as Thermus aquaticus (Taq) DNA polymerase. A typical three-step PCR protocol (see Innis et al., Chapter 1) may include denaturation, or strand melting, at 93-95 degrees C. for more than 5 sec, primer annealing at 55-65 degrees C. for 10-60 sec, and primer extension for 15-120 sec at a temperature at which the polymerase is highly active, for example, 72 degrees C. for Taq DNA polymerase. A typical two-step PCR protocol may differ by having the same temperature for primer annealing as for primer extension, for example, 60 degrees C. or 72 degrees C. For either three-step PCR or two-step PCR, an amplification involves cycling the reaction mixture through the foregoing series of steps numerous times, typically 25-40 times. During the course of the reaction the times and temperatures of individual steps in the reaction may remain unchanged from cycle to cycle, or they may be changed at one or more points in the course of the reaction to promote efficiency or enhance selectivity. In addition to the pair of primers and target nucleic acid a PCR reaction mixture typically contains each of the four deoxyribonucleotide 5′ triphosphates (dNTPs) at equimolar concentrations, a thermostable polymerase, a divalent cation, and a buffering agent. A reverse transcriptase is included for RNA targets, unless the polymerase possesses that activity. The volume of such reactions is typically 25-100 μl. Multiple target sequences can be amplified in the same reaction. In the case of cDNA amplification, PCR is preceded by a separate reaction for reverse transcription of RNA into cDNA, unless the polymerase used in the PCR possesses reverse transcriptase activity. The number of cycles for a particular PCR amplification depends on several factors including: a) the amount of the starting material, b) the efficiency of the reaction, and c) the method and sensitivity of detection or subsequent analysis of the product. Cycling conditions, reagent concentrations, primer design, and appropriate apparatuses for typical cyclic amplification reactions are well known in the art (see, for example, Ausubel, F. Current Protocols in Molecular Biology (1988) Chapter 15: “The Polymerase Chain Reaction,” J. Wiley (New York, N.Y. (USA)).

Ideally, each strand of each amplicon molecule binds a primer at one end and serves as a template for a subsequent round of synthesis. The rate of generation of primer extension products, or amplicons, is thus generally exponential, theoretically doubling during each cycle. The amplicons include both plus (+) and minus (−) strands, which hybridize to one another to form double strands. To differentiate typical PCR from special variations described herein, typical PCR is referred to as “symmetric” PCR. Symmetric PCR thus results in an exponential increase of one or more double-stranded amplicon molecules, and both strands of each amplicon accumulate in equal amounts during each round of replication. The efficiency of exponential amplification via symmetric PCR eventually declines, and the rate of amplicon accumulation slows down and stops. Kinetic analysis of symmetric PCR reveals that reactions are composed of: a) an undetected amplification phase (initial cycles) during which both strands of the target sequence increase exponentially, but the amount of the product thus far accumulated is below the detectable level for the particular method of detection in use; b) a detected amplification phase (additional cycles) during which both strands of the target sequence continue to increase in parallel and the amount of the product is detectable; c) a plateau phase (terminal cycles) during which synthesis of both strands of the amplicon gradually stops and the amount of product no longer increases. Symmetric reactions slow down and stop because the increasing concentrations of complementary amplicon strands hybridize to each other (reanneal), and this out-competes the ability of the separate primers to hybridize to their respective target strands. Typically reactions are run long enough to guarantee accumulation of a detectable amount of product, without regard to the exact number of cycles needed to accomplish that purpose.

A technique that has found limited use for making single-stranded DNA directly in a PCR reaction is “asymmetric PCR.” Gyllensten and Erlich, “Generation of Single-Stranded DNA by the polymerase chain reaction and its application to direct sequencing of the HLA-DQA Locus,” Proc. Natl. Acad. Sci. (USA) 85: 7652 7656 (1988); Gyllensten, U. B. and Erlich, H. A. (1991) “Methods for generating single stranded DNA by the polymerase chain reaction” U.S. Pat. No. 5,066,584, Nov. 19, 1991; all of which are herein incorporated by reference. Asymmetric PCR differs from symmetric PCR in that one of the primers is added in limiting amount, typically 1/100th to ⅕th of the concentration of the other primer. Double-stranded amplicon accumulates during the early temperature cycles, as in symmetric PCR, but one primer is depleted, typically after 15-25 PCR cycles, depending on the number of starting templates. Linear amplification of one strand takes place during subsequent cycles utilizing the undepleted primer. Primers used in asymmetric PCR reactions reported in the literature, including the Gyllensten patent, are often the same primers known for use in symmetric PCR. Poddar (Poddar, S. (2000) “Symmetric vs. Asymmetric PCR and Molecular Beacon Probe in the Detection of a Target Gene of Adenovirus,” Mol. Cell. Probes 14: 25 32 compared symmetric and asymmetric PCR for amplifying an adenovirus substrate by an end-point assay that included 40 thermal cycles. He reported that a primer ratio of 50:1 was optimal and that asymmetric PCR assays had better sensitivity that, however, dropped significantly for dilute substrate solutions that presumably contained lower numbers of target molecules. In some embodiments, asymmetric PCR is used with embodiments of the assays described herein.

In certain embodiments, an amplification method is used that is known as “Linear-After-The Exponential PCR” or, for short, “LATE-PCR.” LATE-PCR is a non-symmetric PCR method; that is, it utilizes unequal concentrations of primers and yields single-stranded primer-extension products, or amplicons. LATE-PCR includes innovations in primer design, in temperature cycling profiles, and in hybridization probe design. Being a type of PCR process, LATE-PCR utilizes the basic steps of strand melting, primer annealing, and primer extension by a DNA polymerase caused or enabled to occur repeatedly by a series of temperature cycles. In the early cycles of a LATE-PCR amplification, when both primers are present, LATE-PCR amplification amplifies both strands of a target sequence exponentially, as occurs in conventional symmetric PCR. LATE-PCR then switches to synthesis of only one strand of the target sequence for additional cycles of amplification. In certain real-time LATE-PCR assays, the limiting primer is exhausted within a few cycles after the reaction reaches its C_{T} value, and in the certain assays one cycle after the reaction reaches its C_{T} value. As defined above, the C_{T} value is the thermal cycle at which signal becomes detectable above the empirically determined background level of the reaction. Whereas a symmetric PCR amplification typically reaches a plateau phase and stops generating new amplicons by the 50th thermal cycle, LATE-PCR amplifications do not plateau and continue to generate single-stranded amplicons well beyond the 50th cycle, even through the 100th cycle. LATE-PCR amplifications and assays typically include at least 60 cycles, preferably at least 70 cycles when small (10,000 or less) numbers of target molecules are present at the start of amplification.

With certain exceptions, the ingredients of a reaction mixture for LATE-PCR amplification are generally the same as the ingredients of a reaction mixture for a corresponding symmetric PCR amplification. The mixture typically includes each of the four deoxyribonucleotide 5′ triphosphates (dNTPs) at equimolar concentrations, a thermostable polymerase, a divalent cation, and a buffering agent. As with symmetric PCR amplifications, it may include additional ingredients, for example reverse transcriptase for RNA targets. Non-natural dNTPs may be utilized. For instance, dUTP can be substituted for dTTP and used at 3 times the concentration of the other dNTPs due to the less efficient incorporation by Taq DNA polymerase.

In certain embodiments, the starting molar concentration of one primer, the “Limiting Primer,” is less than the starting molar concentration of the other primer, the “Excess Primer.” The ratio of the starting concentrations of the Excess Primer and the Limiting Primer is generally at least 5:1, preferably at least 10:1, and more preferably at least 20:1. The ratio of Excess Primer to Limiting Primer can be, for example, 5:1 . . . 10:1, 15:1 . . . 20:1 . . . 25:1 . . . 30:1 . . . 35:1 . . . 40:1 . . . 45:1 . . . 50:1 . . . 55:1 . . . 60:1 . . . 65:1 . . . 70:1 . . . 75:1 . . . 80:1 . . . 85:1 . . . 90:1 . . . 95:1 . . . or 100:1 . . . 1000:1 . . . or more. Primer length and sequence are adjusted or modified, preferably at the 5′ end of the molecule, such that the concentration-adjusted melting temperature of the Limiting Primer at the start of the reaction, T_{M[}0]^{L}, is greater than or equal (plus or minus 0.5 degrees C.) to the concentration-adjusted melting point of the Excess Primer at the start of the reaction, T_{M[}0]^{X}. Preferably the difference (T_{M[}0]^{L}-T_{M[}0]^{X}) is at least +3, and more preferably the difference is at least +5 degrees C.

Amplifications and assays according to embodiments of methods described herein can be performed with initial reaction mixtures having ranges of concentrations of target molecules and primers. LATE-PCR assays are particularly suited for amplifications that utilize small reaction-mixture volumes and relatively few molecules containing the target sequence, sometimes referred to as “low copy number.” While LATE-PCR can be used to assay samples containing large amounts of target, for example up to 10⁶ copies of target molecules, other ranges that can be employed are much smaller amounts, from to 1-50,000 copies, 1-10,000 copies and 1-1,000 copies. In certain embodiments, the concentration of the Limiting Primer is from a few nanomolar (nM) up to 200 nM. The Limiting Primer concentration is preferably as far toward the low end of the range as detection sensitivity permits.

As with PCR, either symmetric or asymmetric, LATE-PCR amplifications include repeated thermal cycling through the steps of strand melting, primer annealing and primer extension. Temperatures and times for the three steps are typically, as with symmetric PCR, 93-95 degrees C. for at least 5 sec for strand melting, 55-65 degrees C. for 10-60 sec for annealing primers, and 72 degrees C. for 15-120 sec for primer extension. For 3-step PCR amplifications, primer annealing times are generally in the range of 10-20 sec. Variations of temperature and time for PCR amplifications are known to persons skilled in the art and are generally applicable to LATE-PCR as well. For example, so-called “2-step” PCR, in which one temperature is used for both primer annealing and primer extension, can be used for LATE-PCR. In the case of “2-step” reactions the combined annealing-extension step can be longer than 30 sec, but preferably as short as possible and generally not longer that 120 sec.

Design of primer pairs for use in LATE-PCR can be performed directly, as will be explained. Alternatively, it can begin with selecting or designing a primer pair for symmetric PCR by known methods, followed by modifications for LATE-PCR. In general, symmetric PCR primers are designed to have equal melting points at some set of standard conditions of primers concentration and salt concentration. Symmetric PCR primers are conveniently designed and analyzed utilizing an available computer program. For symmetric and asymmetric PCR the standard techniques for calculating melting temperatures (T_{M}) have been the “Nearest Neighbor” method and the “2(A+T)+4(G+C)” method. As discussed above, T_{M[}1] which is the T_{M} of the primer at a standard primer concentration of 1 uM and 0.07M salt (monovalent cations). Conversion from the T_{M} given by a typical computer program to T_{M[}1] generally has minimal effect on the relationship of the T_{M}'s of a primer pair. For the concentration-adjusted melting temperatures of primer pairs in embodiments described herein, either actual measurement or an appropriate calculation is generally required.

In practice, once a particular target sequence (for instance a sequence flanking a polymorphism within a chromosomal region of interest) has been chosen for amplification, several candidate pairs of equal T_{M} primers are designed via a computer program such as Visual OMP®. The candidate primer pairs can then be scrutinized on the basis of additional criteria, such as possible primer-dimer formation, that are known in the art to cause non-desirable primer qualities. Satisfactory pairs of candidate primers are further scrutinized using software such as “Blast” for possible non-specific matches to DNA sequences elsewhere in the known genome from the species of the target sequence (Madden, T. L. et al. (1996) “Applications of Network BLAST Server,” Meth. Enzymol. 266: 131 141). Primers pairs are then compared as to their T_{M[}0] values at several different possible concentrations and ratios such that the primer chosen to be the Limiting Primer will have an equal or greater T_{M[}0] relative to the primer chosen to be the Excess Primer. In addition, pairs of candidate primers are examined in relation to the sequence of the amplicon they are expected to generate. For instance, certain target sequences may contain a GC-rich sequence at one end and a less GC-rich sequence at the other end. Where that occurs, choosing the Limiting Primer sequence within sequences at the GC-rich end will assist in achieving a higher melting point for the Limiting Primer relative to the Excess Primer, which will consist of sequences in the less GC-rich end. Examination of the candidate primer pairs relative to the amplicon sequence may suggest additional or different ways of modifying the sequences of one or both members of the pair, such as deliberately increasing or decreasing the length of the primer, most preferably at its 5′ end, or introducing changes in base sequences within the primer which deliberately cause it to mismatch with its target in small regions. Such changes will increase or decrease the T_{M[}0] of either the Limiting or Excess primer.

### II. 3T LATE-PCR

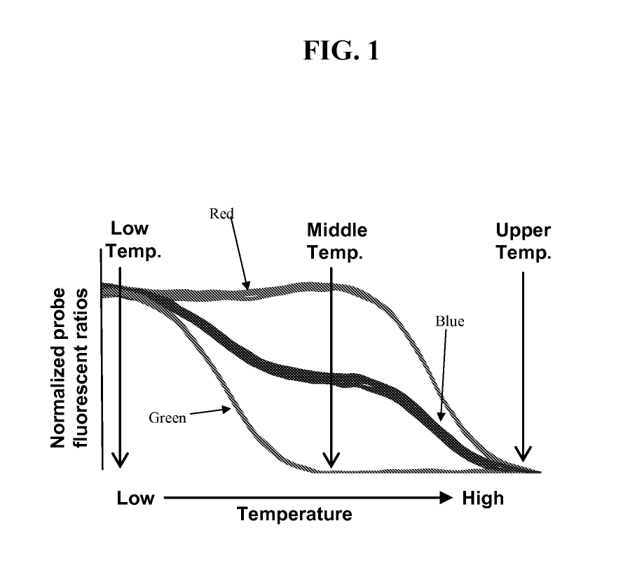

In some embodiments, the present invention utilizes Three Temperature (3T) LATE-PCR. 3T LATE-PCR endpoint assays work by amplifying a segment of genomic DNA containing a polymorphism using a single pair of LATE-PCR primers and then using a single mismatched-tolerant probe that is perfectly complementary to the nucleotide position of one of the alleles and mismatched to the other allelic variants to interrogate the amplified single stranded DNA products at three specific temperatures below the annealing temperature of the PCR (low-Tm probes). The upper temperature is chosen to be too high for the probe to hybridize to either target and therefore provides a measure of background fluorescence. The middle temperature is the temperature at which there is the greatest difference between the amount of probe hybridized to the targets matched at the site of the complementary allele from those targets that are mismatched at the same allelic site. This temperature is derived from analysis of the temperature window of allele discrimination of the mismatch-tolerant probe (see FIG. 1). The low temperature used for 3T-LATE-PCR endpoint assays is chosen as a temperature at which the mismatch-tolerant probe hybridizes at least 90% (e.g., 100%) to all the allelic SNP target sequences, including those mismatched to the probe, see FIG. 1. The signals at the low and upper temperature are then used to normalize the fluorescent signals obtained at the middle temperature.

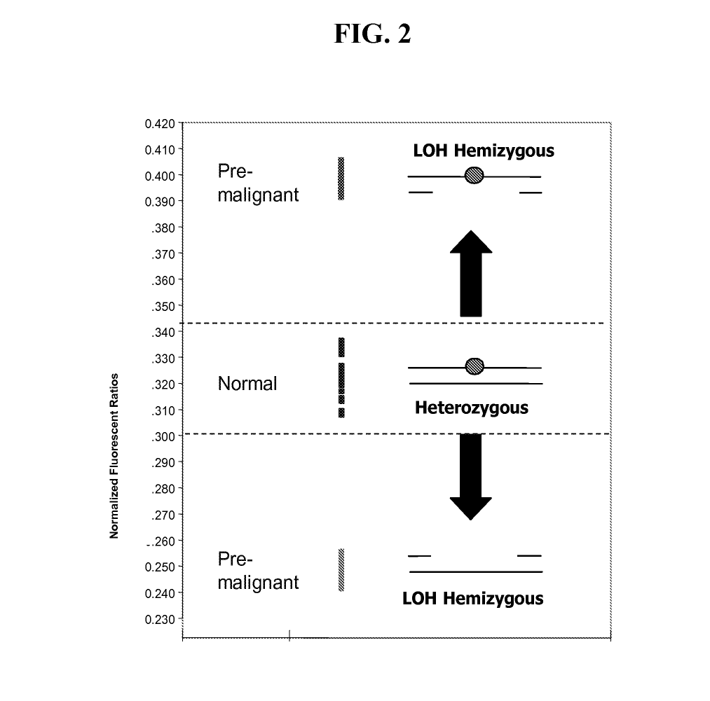

The normalized signal at the middle temperature reveals whether the polymorphic alleles sites (the one perfectly complementary and the one mismatched to the probe), when present, vary in relative abundance in the interrogated genome when compared to genomes heterozygous for the same alleles. Such allele imbalances are indicative of chromosome duplications or deletions in such genome. In intact heterozygous genomes (e.g., carrying equal amounts of both polymorphic alleles), the specific polymorphic allele perfectly complementary to the probe comprises 50% of total amplified single-strands at the mid-temperature and to 100% of total single-strands at a lower temperature, assuming perfect allele-discrimination and no effect of temperature on fluorescent intensities. However, if the polymorphic site in question is deleted or duplicated the fraction single stranded DNA products containing this particular polymorphic allele detected by the probe at the mid temperature will change relative to the total number of single strands detected at the lower temperature. In the case of a deletion, if the perfectly-complementary allele persists the probe detects the same number of single-stranded molecules at both the mid-temperature and the lower temperature. In contrast, if the perfectly-complementary allele is lost and the partially-complementary allele retained, the probe detects little or none of the single-stranded molecules at the mid-temperature but all amplified single-stranded molecules at the lower temperature. The ratio for the normalized signals at the mid-temperature shifts up or down from the heterozygous value in accord with which allele has been lost or duplicated (see FIG. 2 for an specific example of loss of a polymorphic allele by deletion, a phenomenon known a loss of heterozygosity, LOH). Similarly, in the case of a duplication, the ratio for the normalized signals at the mid-temperature shifts up or down from the heterozygous value depending on whether the perfectly complementary allele has been duplicated or not.

### III. Probe Design for Asymmetric PCR

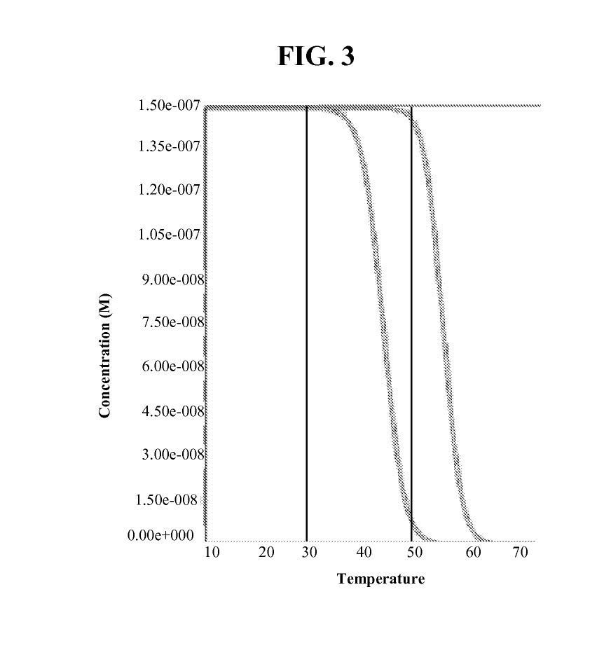

In some embodiments, the present invention provides methods for optimizing probe design for asymmetric PCR (e.g., 3T-LATE-PCR). Embodiments of the present invention describe the specific melting profile of the mismatch-tolerant low T_{M} probe binding to the target that is perfectly matched at the site of the complementary polymorphic allele and to the target that is mismatched at the site of the other polymorphic allele to obtain maximum sensitivity for detection of allelic imbalances (see FIG. 3—the term VOMP refers to the software program used to calculate the probe melting profile). As elaborated further below, maximal detection sensitivity is achieved when the mismatch-tolerant probe binds at least 90% (e.g., 100%) of the target with the complementary polymorphic allele and to less than 10% (e.g., 0%) of the target with the mismatched polymorphic allele at the middle temperature in silico and on synthetic test targets. In some embodiments, the mismatch-tolerant probe binds to 100% of the targets with the matched and mismatched polymorphic alleles at the low temperature (typically between 30 and 40° C.) in silico and on synthetic test targets.

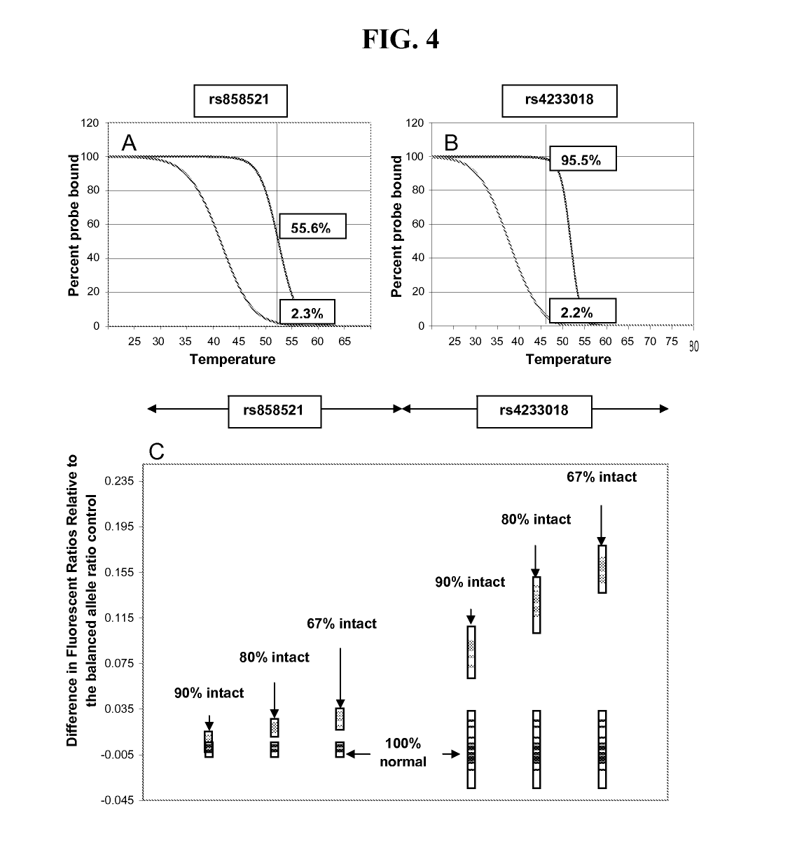

FIG. 4 illustrates the advantages of using mismatch-tolerant probes designed according to the criteria described herein. This figure compares two mismatch-tolerant probes, one for SNP rs858521 and the other for SNP rs4233018 near the TP53 gene in human chromosome 17, for their ability to detect loss of one of the interrogated SNP alleles in the presence of increasing numbers of intact genomes heterozygous for the SNP site. These detection probes differ in the numbers of single-stranded LATE-PCR products containing the corresponding perfectly complementary SNP allele that the probes detect at the mid temperature used for endpoint analysis (i.e., the temperature at which the probe is most allele-discriminating, e.g., the probe binds the most numbers of LATE-PCR products with the perfectly complementary SNP allele the least numbers of the LATE-PCR products with the mismatched SNP allele). Thus, the rs858521 probe hybridizes to only 55.6% of its targets with the matched SNP allele at its corresponding mid-temperature (FIG. 4A). In contrast, the rs4322018 probe, designed according to the criteria described herein, hybridizes to 95.5% of the targets with the matched SNP allele and less than 3% of the targets with the mismatched SNP alleles at its corresponding mid-temperature (FIG. 4B). Both probes hybridize to 100% of the targets with the matched and mismatched SNP alleles at their corresponding low temperature used for endpoint analysis. FIG. 4C compares the effect of these differences in probe design criteria for analysis of artificial mixtures of genomic DNA that simulate genomes deleted for one SNP allele at either the rs858521 or the rs4322018 SNP sites in the presence of 67%, 80%, and 90% excess genomes heterozygous for these SNP sites. A mixture comprised of equal numbers of genomic DNA homozygous for each SNP allele served as 100% heterozygous control. Twenty replicates of each of these DNA mixtures were amplified for either the rs858521 or the rs4322018 sites in the presence of their respective mismatch-tolerant probe. After LATE-PCR amplification, the fluorescent intensities at the three temperatures optimal for each probe were used to calculate the normalized fluorescent ratios for each replicate. These ratios, in turn, were used to define the 99.7% confidence interval for the fluorescent ratios unique to the allele imbalances in each DNA mixture. As seen in FIG. 4C, only the probe designed according to the criteria of embodiments of the present invention (rs43221018 probe) readily distinguished the DNA mixture with 90% normal genomes, 10% LOH genomes from the 100% heterozygous control as evidenced by the lack of overlap between the 99.7% confidence intervals for the fluorescent ratios for these DNA mixtures.

In some embodiments, mismatch-tolerant probes used in 3T-LATE-PCR endpoint assays comprise a linear oligonucleotide carrying a fluorophore and a quencher and modified at the 5′ end to prevent primer-independent degradation of the probe in probe-target hybrids by the 5′-3′ exonuclease activity of the polymerase used for PCR amplification (e.g., Taq DNA polymerase or other suitable polymerases). In some embodiments, the 5′ modification is a nuclease resistant fluorophore (i.e., Cy5) or the quencher. If the fluorescent moiety at the 5′ end is sensitive to exonuclease activity as it is the case for FAM and HEX, the fluorescent moiety resides at the 3′ end of the probe oligonucleotide. In other embodiments, the 5′ end modification comprises a hairpin structure non-complementary to the target comprising a 3 nucleotide loop, a 4-6 nucleotide stem, and a 5′ fluorophore or quencher (independently of whether the fluorophore or the quencher provide resistance to exonuclease activity). In order to reduce probe background signals in the unbound state for a completely linear mismatch-tolerant probe, the probe may be redesigned such that one or two of the terminal nucleotides at the 3′ and 5′ ends are complementary to each other in order to further stabilize the interaction between the fluorophore and the quencher on the probe in the unbound state. In some embodiments, mismatch-tolerant probes are labeled with a 5′ Black Hole Quencher (BHQ-1) and a 3′ fluorescein (FAM).

Mismatch-tolerant probes that meet the criteria for maximal assay resolution typically exhibit an 8° C.-10° C. temperature window of allele discrimination based solely on a single nucleotide difference between the targets with the matched and mismatched polymorphic allele. In some embodiments, Visual OMP (DNA Software, Ann Arbor, Mich.) is used for software design. This program readily generates linear probes that meet these criteria for ˜70% of polymorphic sites examined to date. For the remaining SNP sites, three strategies were developed to preferentially destabilize the probe-mismatched target hybrid and achieve the desired temperature window of allele discrimination: (1) addition of a nucleotide “tail” of least two nucleotides that are not complementary to the target at either the 5′ end, at the 3′ end, or at both ends of the probe generates dangling end(s) that can destabilize probe hybridization to mismatched targets, (2) placing a mismatched nucleotide one nucleotide away from the position of the polymorphic allele on the probe to generate at least a three-nucleotide mismatched “bubble” upon hybridization of the probe to the mismatched target, (3) placing the polymorphic site 2-3 nucleotides from either the 5′ end or the 3′ end of the probe to allow the mismatched polymorphic allele to create a “wedge” that destabilizes binding of the terminal nucleotides in probe-mismatched target hybrids.

### IV. Non-Amplifiable Control Targets.

In some embodiments, the present invention provides non-amplifiable controls targets for use in asymmetric PCR (e.g., 3T LATE-PCR). In some embodiments, the non-amplifiable control oligonucleotides are added to the DNA sample to be tested prior to amplification to generate a reference fluorescent signal or reference fluorescence signal ratios for signal normalization or for genotype and copy number evaluation at the interrogated polymorphic site prior to the start of PCR. In some embodiments, the reference fluorescent signal generated before amplification is used to normalize fluorescent signals generated in the same sample after amplification to correct for variation in latter signals among replicate samples. In the case of fluorescent signal ratios, comparison of the fluorescent ratio from the non-amplifiable control targets prior to PCR and the fluorescent ratios obtained from the amplification products of the test DNA sample at the end of PCR reveals whether or not the tested DNA sample is heterozygous and/or diploid at the interrogated polymorphic site. The post-PCR fluorescent ratios are obtained from adjusted fluorescent signals obtained by subtracting the pre-PCR fluorescent signals from the non-amplifiable control targets from the total fluorescent signals obtained at the end of PCR resulting from PCR products and the non-amplifiable control targets. This approach circumvents the need for external DNA controls of known genotypes for the interrogated polymorphic site to generate reference fluorescence ratios for genotype and copy number evaluation and increases intra-assay as well as inter-assay reliability.

In some embodiments, the non-amplifiable control target comprises or consists of an equimolar mixture of two target oligonucleotides, each one containing the binding site for the mismatch-tolerant probe used in the assay and a different allele of the polymorphic site interrogated by the probe.

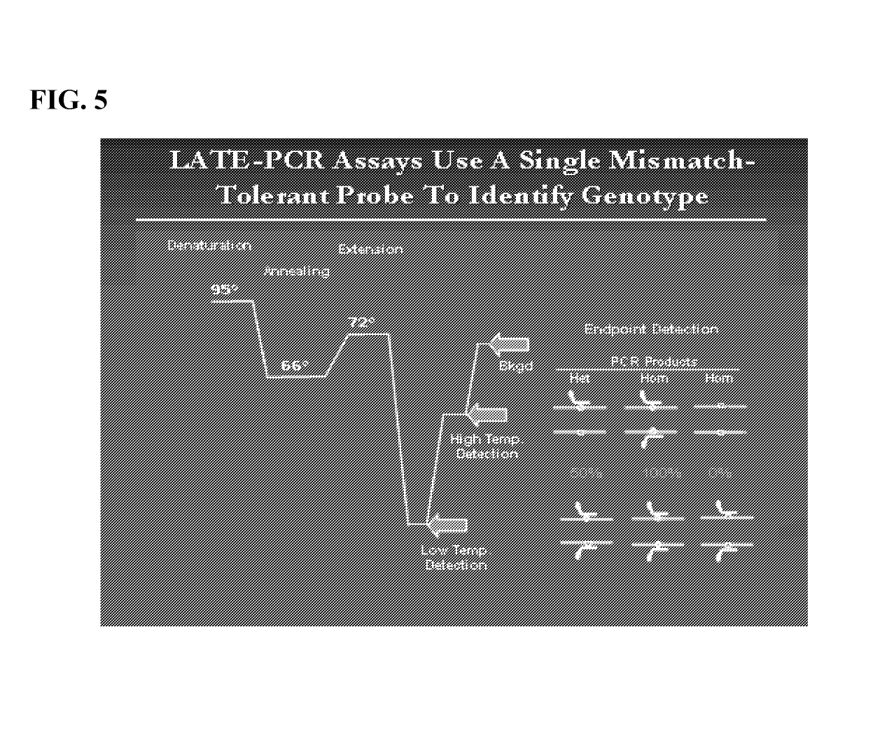

In some embodiments, 3T LATE-PCR endpoint genotyping uses a single mismatch-tolerant fluorescent probe to measure the fraction of one of the polymorphic alleles associated with each genotype. LATE-PCR generates large amounts of single-stranded DNA products that remain available for detection with the mismatch-tolerant probe over a large range of temperatures at the end of the amplification reaction. The single mismatch-tolerant probe comprises or consists of a linear oligonucleotide (e.g., labeled with a fluorophore at one end and a quencher at the other end) and designed to bind below the annealing temperature of the PCR (low-Tm probes). The probe is specifically constructed such that it binds exclusively to the totality of targets with the polymorphic allele site perfectly complementary to the probe at a high temperature and to the totality of the polymorphic allele variants of the same target at a sufficiently low temperature. The ratio of fluorescence signals at these two temperatures corrected for background probe signals collected at a third temperature where the probe does not bind to either allele target reflects the fraction of the interrogated polymorphic allele in the sample and represents molecular signatures unique to each genotype. Thus, following normalization for background signal differences among replicates and notwithstanding the quenching effect of temperature on fluorescence signals, heterozygous samples where the interrogated allele corresponds to 50% of the total amplification products generate half the fluorescence signal at high temperature and 100% of the fluorescence signal at the lower temperature. In contrast, homozygous samples comprising 100% of the interrogated allele generate the same fluorescence signal at both high and low temperatures. Samples homozygous for the polymorphic allele that is not interrogated at the high temperature generate no fluorescent signal at high temperature and 100% of the fluorescent signal at the lower temperature, see FIG. 5. Samples with polymorphic allele imbalances due to chromosomal duplications and deletions generate fluorescent signals at the higher temperature that are either larger or smaller than the fluorescent signals from heterozygous samples at the same temperature and 100% of the fluorescent signal at the lower temperature.

LATE-PCR endpoint genotyping assays are robust because the fluorescence signal ratios associated with each genotype are an intrinsic thermodynamic property of the hybridization probe/target pair and are therefore independent of the amount of starting material in the amplification reaction or the amplification cycle chosen for end-point analysis during the linear phase of LATE-PCR. LATE-PCR endpoint genotyping assays also exhibit a greater multiplex capacity because, unlike traditional homogeneous genotyping methods that use fluorescent probes of different color for each allele, LATE-PCR endpoint assays only require a single fluorescent probe of any given color per polymorphic site.

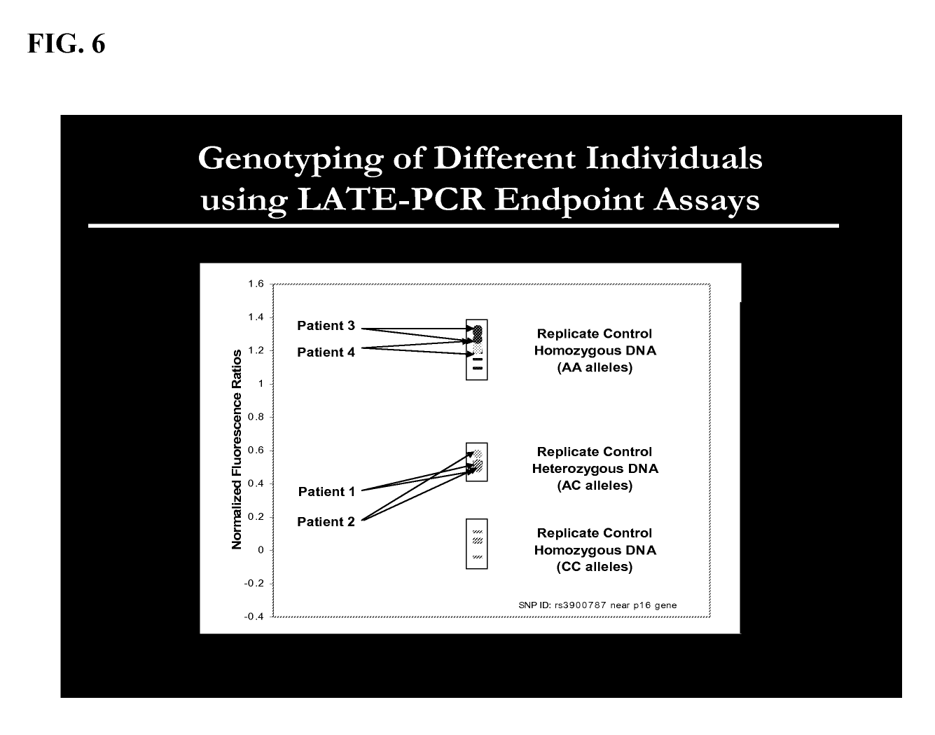

In practice, the fluorescent ratios associated with each genotype are first measured using replicate control DNAs of known genotypes for the polymorphic allele complementary to the probe. To account for slight variations in replicate fluorescent ratios due to sample-to-sample differences, the replicate fluorescent ratios from controls DNA samples are then used to define the 95% confidence interval for the range of fluorescent ratios associated with each genotype. Finally, genotype assignment for an unknown DNA sample is simply performed by measuring the fluorescent ratio of that sample and then determining into which 95% confidence interval of any given genotype the unknown fluorescent ratio falls into (see FIG. 6).

A concern when using the LATE-PCR endpoint genotyping method is the variation in the 95% confidence intervals for the fluorescent ratios associated with each genotype in between experiments. Inter-assay variability of the 95% confidence intervals makes it difficult to define the 95% confidence intervals associated with each genotype in one experiment in advance, and then use those confidence intervals values for genotype assignment of fluorescent ratios from unknown samples in subsequent experiments. As a result, LATE-PCR endpoint genotyping involves determining the 95% confidence intervals for each genotype separately for every experiment. For simplicity, only the 95% confidence interval of the heterozygous genotype is generally determined. Thus, in order to perform an assay with 24 replicate control DNAs to define each 95% confidence intervals, 24 heterozygous DNA control reactions are used in order to genotype a single unknown DNA sample. The non-amplifiable control targets described herein allow for 3T-LATE-PCR genotyping of unknown samples in a single tube, without the need for external reference control samples.

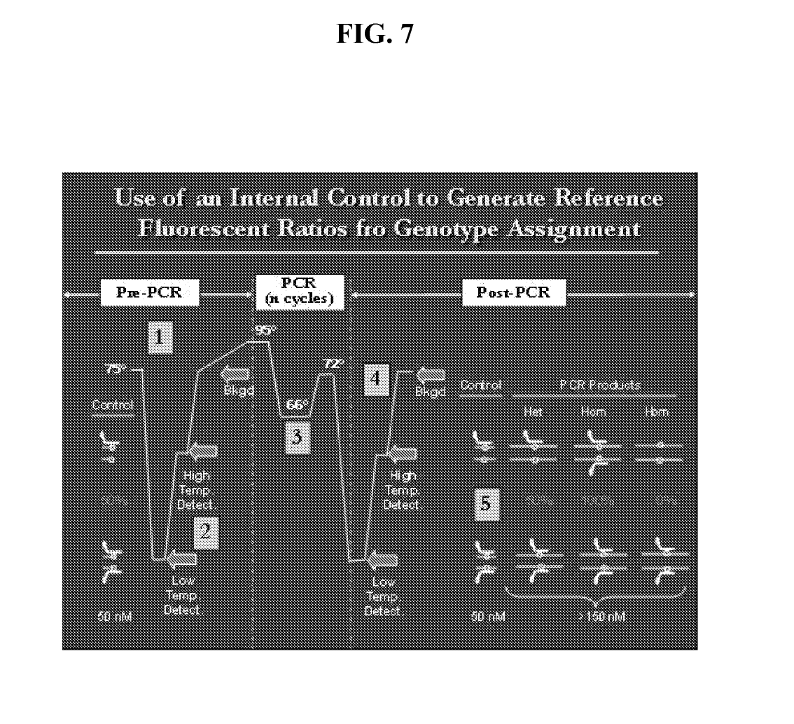

In some embodiments, the non-amplifiable control targets comprises or consists of an equimolar mixture of synthetic oligonucleotides that (1) contain probe targets that are matched and/or the mismatched at the site of the interrogated polymorphic alleles (this does not preclude the probes from being mismatched at sites other that the polymorphic site on the target), (2) lack the binding sites for the primers and it is therefore not amplifiable, and (3) are blocked at their 3′ end and are not complementary to the 3′ end of the amplification primers to prevent them from participating in the amplification reaction. The non-amplifiable control targets are added at low concentrations (e.g., 50 nM) to a DNA sample to be tested prior to amplification to simulate heterozygous control DNA (Step 1, FIG. 7).

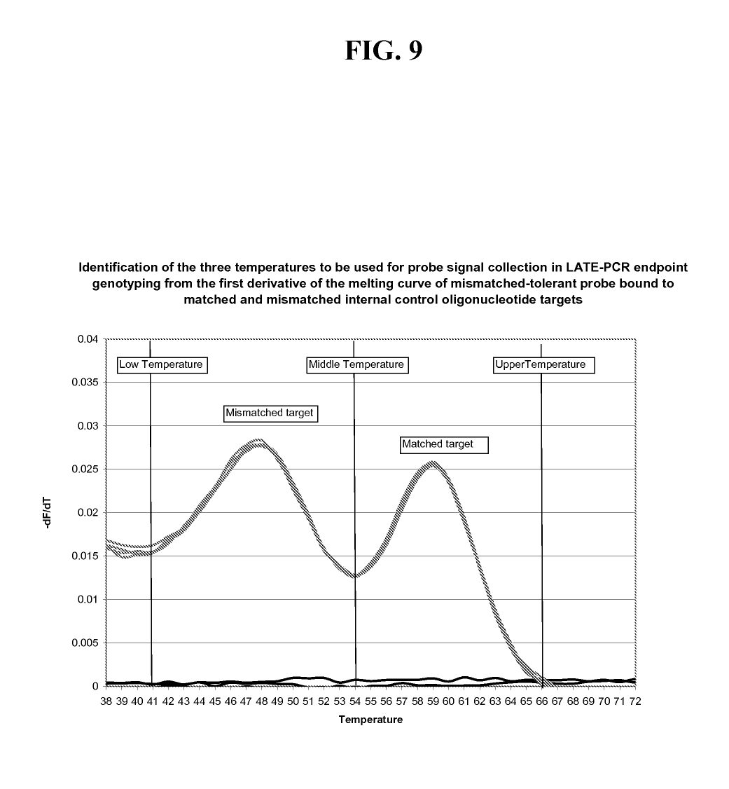

Following binding of the mismatched-tolerant probe to the non-amplifiable allelic control oligonucleotides present in equimolar amounts to simulate the heterozygous genotype, the sample is heated up and fluorescent signals are collected at three different temperatures (Step 2, FIG. 7). These fluorescent signals are then used to determine the pre-PCR fluorescent ratio from the non-amplifiable control targets corresponding to the heterozygous genotype for each particular LATE-PCR sample. The sample is then subjected to LATE-PCR amplification (Step 3—FIG. 7). After PCR, the probe is annealed to the newly generated PCR products and the existing non-amplifiable control targets, heated up and post-PCR fluorescent signals corresponding to the mixture of PCR products and the non-amplifiable control targets are collected (Step 4, FIG. 7). The pre-PCR fluorescent signals collected at a given set of temperatures are then subtracted from the post-PCR fluorescent signals collected at the same set of temperatures to obtain adjusted fluorescent signal derived from exclusively from the PCR products at said temperatures (Step 5, FIG. 7). These post-PCR adjusted fluorescent signals are then used to determine the post-PCR fluorescent ratio from the amplified PCR products. Finally the post-PCR fluorescent ratio is compared to the pre-PCR control fluorescent ratio for genotype assignment. A DNA sample heterozygous for the interrogated polymorphic allele will exhibit post-PCR fluorescent ratios that are approximately equal to the pre-PCR control fluorescent ratios. A DNA sample homozygous for the interrogated polymorphic allele will exhibit post-PCR fluorescent ratios that are larger than the pre-PCR fluorescent ratios from the heterozygous internal control. A DNA sample homozygous for polymorphic allele site that is mismatched to the probe will exhibit post-PCR fluorescent ratios that are smaller than the pre-PCR fluorescent ratios from the heterozygous internal control, see FIG. 9.

The above strategy finds use, for example, because (1) LATE-PCR fluorescent ratios are independent of the amount of target DNA in the reaction. As a result, fluorescent ratios obtained from 50 nM heterozygous non-amplifiable control targets are the same as the fluorescent ratio obtained from the much more abundant PCR products at the end of the reaction (>150 nM); (2) non-amplifiable control oligonucleotides can be constructed such that the fluorescent ratio for these templates matches the fluorescent ratio from amplified heterozygous genomic DNA samples over a range of temperatures, despite differences in size and potential secondary structure between the non-amplifiable control templates and the LATE-PCR amplification products; (3) The fluorescent ratios from non-amplifiable control oligonucleotides before LATE-PCR are not significantly altered following PCR amplification, despite Taq DNA polymerase binding to the double-stranded probe-target hybrids, pH changes caused by pyrophosphate release during PCR, and breakdown of the PCR buffer following multiple cycles of heating and cooling during amplification.

Advantages of the non-amplifiable control target approach include, but are not limited to: (1) This strategy eliminates the need for multiple external DNA controls of known genotype for the interrogated SNP allele to define the 95% confidence intervals for the fluorescent ratios unique to each genotype. Each LATE-PCR sample has a built-in non-amplifiable control target that generates a reference heterozygous fluorescence ratio against which the fluorescent ratio from the PCR products is compared for genotype assignment. (2) Since each sample is normalized against itself, this strategy corrects for difference normally found between replicate samples (i.e. differences associate with different well position in the PCR thermal cycle, use of different tubes, subtle differences in reaction conditions among replicate samples, etc). As a result, this strategy improves intra-assay fluorescent ratio reproducibility. By providing a built-in reference fluorescence ratio this approach also solves problems associated with inter-assay fluorescent ratio variability. Greater reproducibility of fluorescent ratio results in improved resolution of biological phenomena that result in quantitative alteration in fluorescent ratios (such as polymorphic allele imbalances resulting from loss of heterozygosity events).

In some embodiments, the non-amplifiable control target comprises or consists of an equimolar mixture of synthetic oligonucleotides comprising the polymorphic allele targets of the mismatch-tolerant probe and is designed to simulate a heterozygous genomic DNA. The probe is complementary at the site of one of the polymorphic alleles on the target and mismatched at the same site for the other allelic targets. Ideally, the non-amplifiable internal control oligonucleotides generate the same fluorescent ratios as amplification products from genomic DNA encompassing the interrogated polymorphic site. The following criteria govern the design of the non-amplifiable control oligonucleotides in some exemplary embodiments of the invention.

### Design:

1. The non-amplifiable control oligonucleotides should include the target site of the mismatched tolerant probe on the target sequence to be amplified by PCR and may include any number of nucleotides flanking the 5′ end and/or the 3′ end probe target sequence in genomic DNA but not the target sequences for the amplification primers
   a. Differences in the 5′ end or 3′ end target overhangs from the probe-target hybrid as well as differences in secondary structure between the amplicon and the non-amplifiable control targets can cause fluorescent ratios differences between these two types of templates. The goal is to generate a non-amplifiable control template that generates a similar fluorescent ratio as the amplicon containing the interrogated polymorphic site. In most (but likely in not all cases) non-amplifiable controls comprising six nucleotides flanking sequence on either side of the probe target sequence work.
2. The non-amplifiable control oligonucleotides should not have any complementary to the 3′ end of the amplification primers that would result in extension of the primers on the probe sequence.
3. The non-amplifiable internal control oligonucleotides should be blocked at the 3′ end to prevent them from acting as primers.

### EXAMPLES

The following Examples are presented in order to provide certain exemplary embodiments of the assays described herein and are not intended to limit the scope thereof.

### Example 1

### Protocol for the Design of Mismatch-Tolerant Probes for LATE-PCR Endpoint SNP Genotyping

This Example described an exemplary protocol for design of LATE-PCR probes for genotyping single nucleotide polymorphisms (SNPs). This protocol can be implemented via computer program with one or more steps conducted in an automated fashion.
a) Obtain information for the SNP sites in the chromosomal regions of interest from the dbSNP database at Pubmed
b) Make sure that the SNP site is indeed in the chromosome of interest (See Integrated Maps section in the dbSNP database)
2) Transfer the FASTA DNA sequence flanking both sides of the SNP from dbSNP database to Word

Clean up sequence in Word by replacing all the white spaces and paragraph marks with empty spaces.
3) Get the sequence for the DNA strand that corresponds to the excess primer strand generated by LATE-PCR. This is the strand that will be bound by the mismatch-tolerant probe.
   a) If LATE-PCR primers are already designed to amplify the SNP site, locate for the excess primer sequence in the cleaned FASTA sequence
      i) If found, the FASTA sequence corresponds to the excess primer strand. If not, use the reverse complement of the FASTA sequence as the excess primer strand.
      ii) Highlight the position of the limiting and the excess primers or their complementary sequence, as appropriate, on the limiting and excess primer strands. Highlight also the position of the SNP site.
         (1) NOTE: The excess primer strand serves as the target for probe binding, the limiting primer strand contains the probe sequence.
   b) If the PCR primers are not designed yet, choose as the excess primer strand for primer design. The DNA strand that has the least secondary structure is selected for the mismatched allele (see STEPs 4-5 below) at 35° C., 70 nM monovalent cations, 3 mM MgCl₂ within 100 nucleotides surrounding the SNP site as determined in silico using Visual OMP software (DNA Software, Inc, Ann Arbor, Mich., abbreviated as VOMP) or any other similar DNA folding program such as DNA-mfold (Zuker (2003) Nucleic Acids Res. 31 (13), 3406-15).
4) Identify the nucleotides corresponding to the SNP alleles on the excess primer strand
   a) Nucleotides are listed under Submitted Records in the dbSNP database. The FASTA sequence corresponds to the FWD strand and the reverse complement as the RVS strand. Nucleotide information is also listed as an IUPAC ambiguity code in the FASTA sequence (for IUPAC code information see step 6.b.d. below).
5) Identify the specific SNP allele on the excess primer DNA strand that will be used as mismatched allele site to the mismatch-tolerant probe.
   a) The target mismatched at the site of the SNP alleles should exhibit the largest probe-target hybrid instability out of the two possible SNP allele choices. The relative stability of mismatched dideoxynucleotide bases is CG>AT>GG>GT>GA>AA>TC>AC (SantaLucia et al., (2004) Ann Rev Biophys Biomol Struct. 33:415-40). For example, in the case of a G/A SNP site, choose A as the mismatched allele target so that the complementary C nucleotide on the mismatched-tolerant probe creates a very unstable C/A hybrid. If the G allele were to have been chosen as the mismatched target, the complementary T nucleotide on the probe would have created a relatively stable G/T mismatch on the G SNP allele target, which is undesirable.
6) Generate target sequences that are matched and mismatched at the site of the chosen SNP allele from the excess primer strand
   a) Choose a target of ˜20 nucleotides on both sides of the SNP site
7) Generate candidate linear mismatch-tolerant probe sequences for the chosen matched/mismatched allele target using VOMP.
   a) Rational: Since mismatch-tolerant probes are of low-Tm type (e.g., they bind below the PCR annealing temperature in the assay) their Tm is constrained by the Tm of the LATE-PCR primers and the amplification conditions. For LATE-PCR amplification for LOH analysis, the following Tm criteria should apply
      i) Limiting primer Tm (Tm^{L})=71° C.-72° C. @ 50 nM
      ii) Excess primer Tm (Tm^{x})=66° C.-67° C. @ 1000 nM,
         (1) This is according to LATE-PCR primer design criteria (Tm^{L}−Tm^{x}˜5° C.)
      iii) Annealing temperature=63° C.-64° C.
         (1) Rule of thumb is 2° C.-3° C. below the excess primer Tm
      iv) Mismatch-tolerant probe Tm for the matched target ≦62° C.-63° C. @ 500 nM and 150 nM probe and target concentrations, respectively.
         (1) Rule of thumb is 8° C.-10° C. below the limiting primer Tm to prevent the probe from interfering with the extension of the limiting primer.
         (2) This criterion maximizes the temperature space for mismatch-tolerant probe detection.
      v) Mismatch-tolerant probe Tm for the mismatched target ˜52° C.-54° C. but ideally no lower than 45° C. @ 500 nM probe and 150 nM target concentrations
         (1) Rule of thumb is least 8° C.-10° C. below the Tm of the probe-matched target hybrid. This allows the probe to bind to completion to the matched allele before binding to the mismatched allele (see below)
         (2) The higher the temperature for probe binding to its mismatched target the smaller is the likelihood of secondary structure on the target interfering with probe binding.
   b) Steps for generating and evaluating candidate Mismatch-tolerant probe sequences:
      i) Open VOMP
      ii) From the Sequence tab
         (1) Input the amplicon sequence from the excess primer DNA strand identified above.
            (a) Make sure that the matched allele is listed on this sequence
         (2) Name the sequence as amplicon
         (3) Input also the matched and mismatched targets (regions of 20 nucleotides encompassing the corresponding SNP site allele in the excess primer strand).
         (4) Name these sequences as matched and mismatched targets
         (5) For all the sequences, specify 150 nM concentration, single stranded DNA, target
         (6) Under variation tab for the amplicon sequence
            (a) Name=SNP
            (b) Position: from 5′ end including SNP using Word counting function NOT VOMP's
            (c) Type=Substitution
            (d) Sequence=The input sequence is the IUPAC ambiguity code
            (i) R A,G
            (ii) Y C,T
            (iii) M A,C
            (iv) K G,T
            (v) S C,G
            (vi) W A,T
            (e) Length=0
            (f) Then look at the amplicon sequence in the text editor (right click on the sequence and choose “Edit in Text Box”. Double click on the position of the variation to highlight it in the sequence and make sure that the right nucleotide is highlighted.
         (7) >run mock experiment (green triangle) in VOMP to complete data input.
      iii) From the Experimental Conditions tab specify 42° C. assay temperature, 0.07 M monovalent cation, 0.003 M MgCl2
         (1) Run experiment again in VOMP to look at 2^{nd} structure of the amplicon target. Check to see whether the nucleotide range around the SNP allele (˜20 nucleotides) is free of secondary structure. If not, increase the temperature at the Experimental Condition tab (typically to 46° C. or 48° C.) and keep checking the secondary structure until one finds the minimum temperature at which the local secondary structure around the SNP site does not occur. This is the temperature that will be used for probe design
            (a) If the temperature that prevents the local secondary structure is within 5° C. or less of the Tm of the probe for the mismatched allele (52-54° C.) the secondary structure may prevent complete probe binding to that allele at low temperatures and another SNP site should be used for the assay
            (b) Notice the range of nucleotides free of secondary structure in the vicinity of the SNP
      iv) From the Probe/Primer tab
         (1) Design name=Probe #
         (2) Design type=probe
         (3) Target=Amplicon
         (4) Variation=the SNP defined above
         (5) Position Range=10 nucleotides on both sides of the SNP site or the region within the section that is free of secondary structure
         (6) Tm=leave empty (it will be filled automatically from the strategy criteria)
         (7) Length=leave empty (it will be filled automatically from the strategy criteria)
         (8) Modification: BHQ-1 at position 1, FAM (or the appropriate fluor) at position 15 (this will be adjusted later to the specific probe)
         (9) [Oligo]=500
         (10) Units=nM
         (11) Strategy=LOH probe design

| | MIN | OPT | MAX | LT WT | GT WT | WT SCALE | | Value |
|---|---|---|---|---|---|---|---|---|
| SIZE | 10 | 15 | 20 | 1 | 1 | LINEAR | NUM REDUNDANT | 100 |
| DUPLEX TM | 60 | 62 | 63 | 3 | 1 | LINEAR | SOLUTION DISTANCE | 1 |
| DUPLEX ΔG | −100 | −24 | −3 | 0.5 | 1.5 | LINEAR | TGT FOLD LENGTH | 35 |
| LOCAL TGT TM | | 0 | 70 | | 0.5 | LINEAR | MAX POLY A | 10 |
| LOCAL TGT ΔG | −50 | 0 | | 1 | | LINEAR | MAX POLY C | 4 |
| MONOMER TM | | 0 | 65 | | 1 | LINEAR | MAX POLY G | 4 |
| MONOMER ΔG | −50 | 0 | | 1.5 | | LINEAR | MAX POLY T | 10 |
| HOMODIMER TM | | 0 | 65 | | 1 | LINEAR | | |
| HOMODIMER ΔG | −50 | 0 | | 1.5 | | LINEAR | | |
| MISHYB TM | | 52 | 54 | | 0.5 | LINEAR | | |
| MISHYB ΔG | −50 | 0 | | 1 | | LINEAR | | |
| CROSSHYB TM | | 0 | 50 | | 0.5 | LINEAR | | |
| CROSSHYB ΔG | −50 | 0 | | 1 | | LINEAR | | |
| VARIATION POS | 3 | LENGTH/2 | LENGTH-2 | 0.5 | 0.5 | LINEAR | | |
| MAX BLAST HITS | | 0 | 10 | | 1 | LINEAR | | |
| MAX BLAST MATCH LEN | | 0 | 12 | | 0.2 | LINEAR | | |

The table above shows parameters for the strategy “LOH probe design”

| | MIN | OPT | MAX | LT WT | GT WT | WT SCALE | | Value |
|---|---|---|---|---|---|---|---|---|
| SIZE | 10 | 15 | 20 | 1 | 1 | LINEAR | NUM REDUNDANT | 100 |
| DUPLEX TM | 56 | 58 | 60 | 3 | 1 | LINEAR | SOLUTION DISTANCE | 1 |
| DUPLEX ΔG | −100 | −24 | −3 | 0.5 | 1.5 | LINEAR | TGT FOLD LENGTH | 35 |
| LOCAL TGT TM | | 0 | 70 | | 0.5 | LINEAR | MAX POLY A | 10 |
| LOCAL TGT ΔG | −50 | 0 | | 1 | | LINEAR | MAX POLY C | 4 |
| MONOMER TM | | 0 | 65 | | 1 | LINEAR | MAX POLY G | 4 |
| MONOMER ΔG | −50 | 0 | | 1.5 | | LINEAR | MAX POLY T | 10 |
| HOMODIMER TM | | 0 | 65 | | 1 | LINEAR | | |
| HOMODIMER ΔG | −50 | 0 | | 1.5 | | LINEAR | | |
| MISHYB TM | | 48 | 50 | | 0.5 | LINEAR | | |
| MISHYB ΔG | −50 | 0 | | 1 | | LINEAR | | |
| CROSSHYB TM | | 0 | 50 | | 0.5 | LINEAR | | |
| CROSSHYB ΔG | −50 | 0 | | 1 | | LINEAR | | |
| VARIATION POS | 3 | LENGTH/2 | LENGTH-2 | 0.5 | 0.5 | LINEAR | | |
| MAX BLAST HITS | | 0 | 10 | | 1 | LINEAR | | |
| MAX BLAST MATCH LEN | | 0 | 12 | | 0.2 | LINEAR | | |

The table above shows parameters for the strategy “LOH probe design shorter” (see below)
i) From the Design Setting tab, specify Require Solutions
ii) Run Experiment Design (small green triangle with a pencil)
   (1) If no solutions are obtained, try the “LOH probe design shorter” strategy. This strategy generates shorter probes that have lower Tm for the matched alleles (58° C. instead of 62° C.) but are more allele-discriminating
iii) Highlight all the results and right click to “Add to Experiment”
iv) Simulate binding of all probe candidate against the matched and mismatched targets simultaneously
   (1) Right click on the results and select “Export to Excel”
   (2) Select only those rows containing heterodimers between targets and probe and delete the rest of the columns
   (3) Copy the Tm data next to the title of the row and delete the rest of the columns
   (4) Copy the Tm for the mismatched target/probe hybrids and paste them next to the Tm for the matched target/probe hybrids
   (5) Calculate the difference between the matched and mismatched Tm
   (6) Desired probes should have a Tm no greater that 63.5° C. for the matched allele and as high as possible for the mismatched allele (up to 55° C. if possible). The difference between the matched and mismatched Tm should be 7-11 (larger differences are undesirable because they are more difficult to normalize)
   (7) Select candidate sequence for evaluation
   (8) To evaluate each sequence
      (a) Hover the cursor over the row for the candidate sequence and note the sequence length
      (b) Click on the modification tab for that sequence and adjust the position of the fluor modification to match the sequence length
      (c) Make sure that only the matched target, the mismatched target, and the specific probe to be tested are selected (click on the boxes at the start of each row)
      (d) Run the experiment
      (e) Generate a graphic representation of the binding of the probe to each target by clicking on the X icon in the top toolbar. Right click on the graph, select “Select Species” and hide monomer and homodimers and any heterodimer that does not correspond to the matched and mismatched target and probe hybrid.
      (f) The graph should comprise two parallel curves that come together around 40 C (i.e., the C in the graph title) and are sufficiently spaced so the rightmost curve have reached the maximum binding before the leftmost curve does. If not test another candidate probe
         (i) The two curves should come together at least by 35° C. (see FIG. 3)
   (9) In addition, the ideal probe should also bind less than 5% of the mismatched target at a temperature where the probe binds at least 95% of the matched target to permit quantification of the matched alleles without significant contribution from the mismatched allele
      (i) To determine the actual percentage bound for each target, from the concentration vs. temperature plot above, estimate the experimental temperature where the probe is fully bound to the matched target but very little to the mismatched target, run the experiment again at that temperature and check the percentages in the Results. Keep modifying the experimental temperature until the % bound for the matched target is close to 95%.
      (ii) The maximum range allowed is 90% matched to 10% mismatched
v) Once best probe candidate is selected, run against primers and amplicon target (as double stranded DNA) at the annealing temperature (i.e., @64° C.) to check for 3′ end compatibility. Make sure to specify the following concentrations
   (1) Excess primer=1 μM, single stranded primer
   (2) Limiting primer=50 nm, single stranded primer
   (3) EPS=1 nM, double stranded target
   (4) Probe=500 nM single stranded probe.
vi) Identify the selected probe sequence and its complement on the limiting and excess primer strands. Choose as the definite matched and mismatched target sequences the sequence complementary to the probe on the excess primer strand+6 nucleotides on either side.

### Strategies for Achieving Optimal Probe Design:

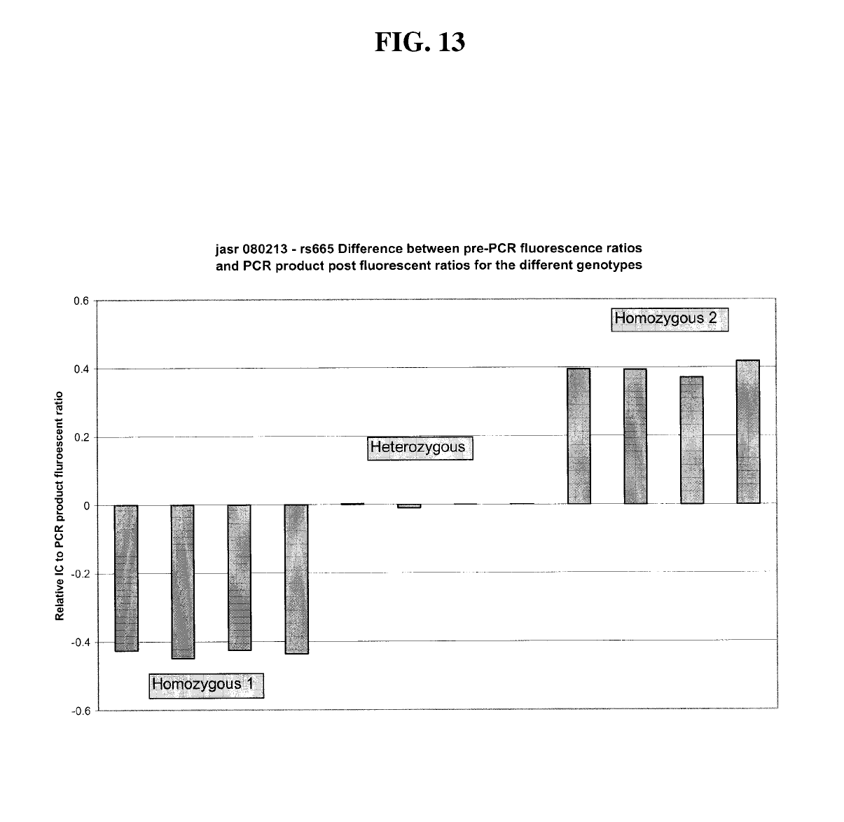

To achieve the above probe design criteria the probe-mismatched target hybrid can be further destabilized as follows (arrows in FIGS. 14A-C indicate the location of the SNP site):

Addition of a 2-3 nucleotide segment not complementary to the target at either the 5′ end, at the 3′ end, or at both ends of the probe generates dangling end(s) that contribute to destabilize further binding of the probe to targets mismatched at the polymorphic site. Introduction of mismatched nucleotides on the probe at least one nucleotides adjacent to the site of the mismatched SNP nucleotide generates a large mismatched “bubble” upon hybridization of the probe to the mismatched target. Placing the SNP site 2-3 nucleotides from either end of the probe allows the mismatched SNP nucleotide to create a “wedge” that destabilizes binding of the terminal nucleotides in probe-target hybrids mismatched at the SNP allelic site.

These criteria are important to improve the resolution of LOH detection in mixtures of LOH-positive and LOH negative cells (see Example 2 below).

Confirm the in silico design criteria by examining the melting profile of the mismatch-tolerant probe on synthetic oligonucleotide targets matched and mismatched at the site of the polymorphic allele in vitro. Failure to reproduce the curves shown in FIG. 3 in vitro, particularly failure of the probe to bind to the totality of target sequence variants indicates the presence of a local secondary structure on the target sequence interfering with probe binding.

### Example 2

### Validation of Design Criteria (1): Optimization of LATE-PCR Endpoint Assays for LOH Detection in Samples Containing Mixtures of Neoplastic and Normal Cells

Detection of LOH genomes can be confounded by the presence of normal diploid genomes from surrounding stromal tissue. To evaluate the resolving power of LATE-PCR endpoint LOH assays for different probe design criteria, artificially constructed mixtures of DNA homozygous for rs858521 SNP (C/G alleles) or the rs4233018 SNP site (A/G alleles), which resulted in different proportions of the SNP alleles were analyzed. The rs858521 probe was not designed to meet the optimal design criteria described above while the rs4233018 was (e.g., only the rs4322018 probe hybridizes to close to 100% of the perfectly matched targets and 0% of the mismatch targets at the mid-temperature while still hybridizing to both target sequences at the low temperature, see FIGS. 4A and 4B). These SNP sites are located in the vicinity of the TP53 tumor suppressor gene in human chromosome 17, a gene that is inactivated in 60%-80% human cancers. Genomes heterozygous for the rs858521 or the rs4322018 SNP sites (50% of each allele) served as a reference. The allele mixtures consisted of 53%-47% allele ratio (3% allele imbalance relative to 50% allele ratio control), 55.5%-44.4% allele ratio (5.5% allele imbalance), and 60%-40% allele ratio (10% allele imbalance). These allele mixtures corresponded to allelic imbalances expected for mixtures of LOH genomes with ˜90%, 80%, and 66.6% excess normal genomes (i.e., 8-fold, 4-fold, and 2-fold excess normal genomes). For this experiment, sets of LATE-PCR primers were constructed for the rs858521 or the rs4322018 SNP locus as well as two mismatched-tolerant mismatch-tolerant probes perfectly complementary to the G allele of each SNP site (see FIG. 4A for the validated design specifications of these probes on synthetic targets). LATE-PCR amplification was carried out in 1×Taq buffer, 3 mM MgCl₂, 250 nM each dNTP, 25 nM 9-22DD Pprimesafe, 500 nM C3 Primesafe, 1.25 units of Taq polymerase, 50 nM limiting primer, 1000 nM excess primer, 500 nM probe. The rs4322018 limiting primer sequence was 5′ CCGTGCCTGGCCAACACAGTATTTAAAAACAA 3′ (SEQ ID NO:1). The rs4322018 excess primer sequence was 5′GTAGAGTACAGTGCTAAGCCATATT 3′ (SEQ ID NO:2). The optimally designed rs4322018 probe sequence was 5′ BHQ1-TACCTTAGGCTCCAATA-FAM 3′ (SEQ ID NO:3). The rs858521 limiting primer sequence was 5′ GCCCAGCCGGTGTCATTTTCTGATCC 3′ (SEQ ID NO:4). The rs858521 excess primer sequence was 5′CAATCCCTTGACCTGTTGTG3′ (SEQ ID NO:5). The sub-optimally designed rs858521 probe sequence was 5′ BHQ1-CTCTTCAGCTCGAACAATAG-FAM 3′ (SEQ ID NO:6). The cycling profile was 95° C. for 3 min, followed by 60 cycles of 95° C. for 10 seconds, 64° C. for 10 seconds, and 72° C. for 20 seconds.

Following LATE-PCR amplification, the tubes were cooled down to 20° C., incubated for 20 minutes to allow for maximal probe binding and fluorescent readings from the hybridization probe were collected at 40° C., 52° C. and 60° C. (for the rs858521 SNP) or at 39° C., 47° C., and 59° C. (for the rs4322018 SNP) for 90 seconds as the mismatch-tolerant probes melted off their target. The resulting data were then used to calculate normalized fluorescent signal ratios (FIG. 4C). The boxes in FIG. 4C correspond to three-standard deviations of the range of fluorescence ratios that define the 99.7% confidence range for each genotype. Only the rs4322018 probe that meet the design criteria described above readily detects allele imbalances as little as 3% (the equivalent to 88.8% normal cells to 11.2% LOH-positive cells, or 8-fold contamination of LOH-positive cells with normal diploid cells, see FIG. 4C). This was not the case for the rs858521 probe, which does not meet the design criteria, where there is significant overlap in the confidence intervals boxes corresponding to the smallest rs858521 allele imbalance (3%) and the rs858521 heterozygous.

### Example 3

### Non-Amplifiable Control Targets

### Pre-PCR Steps

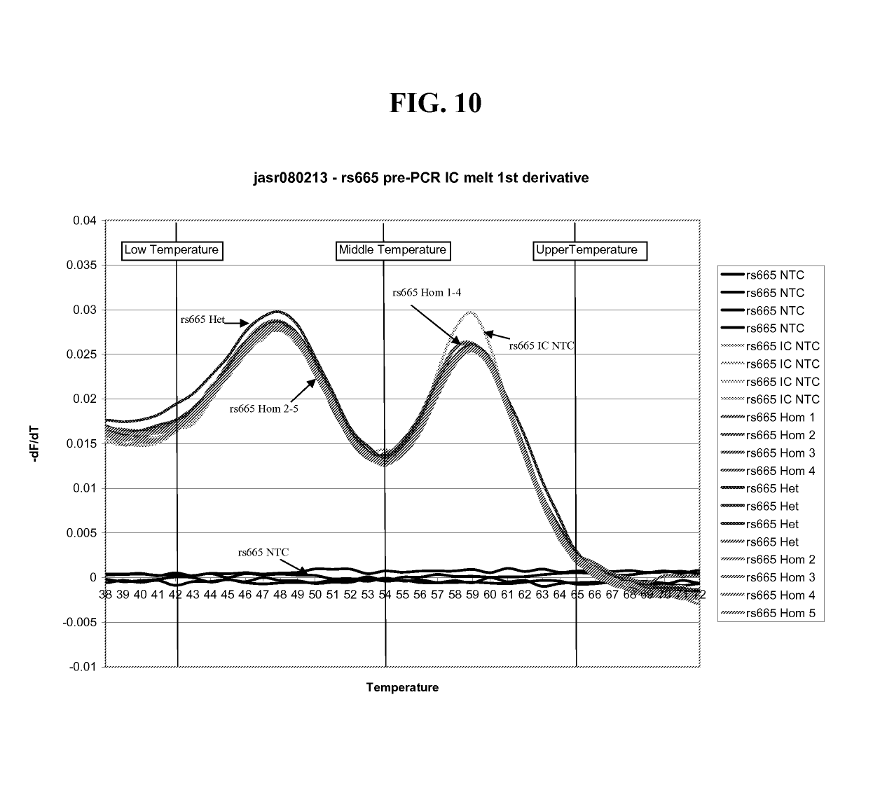

1. The non-amplifiable control oligonucleotides are added to each sample prior to amplification at the lowest concentration that reliably generates fluorescent ratios. The goal is to prevent the control fluorescent signals from overwhelming the fluorescent signals from the PCR products at the end of the reaction. Control experiments showed that 50 nM of each non-amplifiable control oligonucleotides (the same concentration as the typical limiting primer concentration in LATE-PCR) works well.
   a. In addition to the non-amplifiable control targets, the PCR samples contain 1×PCR buffer, MgCl₂, dNTP, primers, probe (500 nM), genomic DNA, and Primesafe (a reagent that prevents primer dimer formation during collection of fluorescent signals from the probe-internal control hybrids at three different temperatures).
2. As shown in FIG. 7, prior to PCR amplification the sample with the control oligonucleotides was first heated to at least 10° C.-15° C. above the Tm of the probe bound to the target that is complementary to one of the polymorphic allele (e.g., a temperature where the probe-control target hybrids are melted but genomic DNA is not denatured yet). The sample is then cooled gradually (0.1° C./sec) to a temperature at least 10° C.-15° C. below the Tm of the probe bound to the target that is mismatched at the site of the other polymorphic allele to allow complete probe target formation.
3. The probe-control target hybrids are then heated up at a fast rate (2°-3° C./sec) at 1° C. intervals 30 seconds long up to at least 10° C.-15° C. above the Tm of the probe bound to target with the allele site complementary to the probe and fluorescent signals are collected at three temperatures. The lowest temperature is highest temperature at which the probe is bound to the totality of all allelic control targets. The middle temperature is the temperature where the probe is bound exclusively to the control targets with the complementary polymorphic allelic site. The upper temperature corresponds to the lowest temperature where the probe is not bound to any non-amplifiable control targets. The actual temperatures to be used are identified from the 1^{st} derivative of the melting curve of probe-control target hybrids, as shown in FIG. 10.
4. Determination of fluorescent signals also after collection of fluorescent signals from samples with probe alone (i.e., with no control oligonucleotide targets) at the same temperatures as above to correct for the effect of temperature on fluorescent signal intensity. The fluorescent ratios from the fluorescent signal collected at these three temperatures are calculated from the formula:

Ratio=[([IC]_{MT}*[Probe alone]_{HT})−([IC]_{HT}*[Probe alone]_{MT})]/[([IC]_{LT}*[Probe alone]_{HT})−([IC]_{HT}*[Probe alone]_{HT})]

### Where:

IC_{LT}=fluorescent signal from the non-amplifiable control at the low temperature.
IC_{MT}=fluorescent signal from the non-amplifiable control at the middle temperature
IC_{HT}=fluorescent signal from the non-amplifiable control at the high temperature
Probe Alone_{LT}=fluorescent signal from the non-amplifiable control at the low temperature.
Probe Alone_{MT}=fluorescent signal from the non-amplifiable control at the middle temperature
Probe Alone_{HT}=fluorescent signal from the non-amplifiable control at the high temperature

### Post-PCR Steps

1. Steps 3-4 above were performed again at the end of the amplification reaction. The pre-PCR control fluorescent signals were subtracted from the post-PCR fluorescent signals on a per temperature basis to obtain the adjusted fluorescent signal values of the PCR products. The resulting adjusted fluorescent signal values were then used to determine the post-PCR fluorescent ratio from the amplified PCR products. The post-PCR product fluorescent signal ratio was then compared to the pre-PCR reference fluorescence signal ratio by subtraction for genotype assignment. If, based on the confidence intervals established from replicate samples, the post-PCR fluorescent signal ratio is identical to the pre-PCR fluorescent signal ratio the sample is then heterozygous for the interrogated SNP allele. If, based on the confidence intervals established from replicate samples, the post-PCR fluorescent signal ratio is greater than the pre-PCR fluorescent signal ratio the sample is then homozygous for the interrogated SNP allele. If, based on the confidence intervals established from replicate samples, the post-PCR fluorescent signal ratio is smaller than the pre-PCR fluorescent signal ratio the sample is then homozygous for the SNP allele that is not perfectly complementary to the mismatch tolerant probe.

### Example 4

### Use of Non-Amplifiable Control Targets in Combination with LATE-PCR Endpoint Assay for Genotyping the rs8066665 SNP Site on Human Chromosome 17

The rs8066665 SNP site consists of an A-to-G polymorphism. DNA samples from individuals of various genotypes for this SNP site (AA genotype—sample NA10855; AG genotype—sample NA10851; GG genotype—sample NA07348) were obtained from the Coriell Cell Repository (Camdem, N.J.). The primers used to amplified genomic DNA encompassing this SNP site were

| | |
|---|---|
| Limiting primer: | |
| 5′ GGAGGTCAGAGTACCCACTGCTCCTTC 3′ | (SEQ ID NO: 7) |
| Excess primer: | |
| 5′ GCTCCTGAGCAATGAGAATGTC 3′ | (SEQ ID NO: 8) |

The mismatched tolerant probe was designed to be perfectly matched to the G allele (Tm=48C) and mismatched to the A allele (Tm=59C)—note these Tm values correspond to 500 nM mismatch-tolerant probe and 50 nM matched or mismatched oligonucleotide targets.

| | |
|---|---|
| SNP rs8066665 C probe: | |
| 5′ BHQ-1 CTTGCAGGTGGGATAGGA FAM 3′ | (SEQ ID NO: 9) |

The synthetic matched and mismatched targets for these probes are

SNP rs8066665 matched target (30 nucleotides)
(SEQ ID NO: 10)
5′ GCATGCTCCTATCCCACCTGCAAGGGGTTG 3′
SNP rs8066665 mismatched target (30 nucleotides)
(SEQ ID NO: 11)
5′ GCATGCTCCTATCCCACCTACAAGGGGTTG 3′

(Note: the highlighted nucleotide corresponds to the SNP allele on the target).
The Tm difference between the matched and mismatched targets allows the probe to bind to 100% of the matched targets before it binds significantly to any mismatched target at a high temperature while also allowing probe binding to both matched and mismatched targets at a sufficiently low temperature.

Twenty LATE-PCR samples comprising 1×PCR buffer, 3 mM MgCl₂, 0.25 mM dNTP, 25 mM PrimeSafe 060, 300 nM PrimeSafe 001, 50 nM limiting primer, 1 μM excess primer, 500 nM mismatched-tolerant C probe, 1.25 units Taq polymerase were prepared.

Sixteen of these samples contained 50 nM each of the matched and the mismatched oligonucleotide targets and the remaining four lacked these synthetic templates and serve as “probe alone” controls. Four of the sixteen samples containing probe plus non-amplifiable control targets alone but no genomic DNA. For the twelve remaining samples with non-amplifiable control templates, sets of four samples received genomic DNA with different genotypes for the rs8066665 SNP site. All these samples were then heated to 72° C. to melt any pre-existing probe-target hybrids and then they were cooled to 30° C. at 0.1° C. per sec to allow formation of probe-target hybrid. The samples were heated up back to 72° C. at 1° C. intervals for 30 seconds each at a rate of 2° C.-3° C./sec. For the purpose of assay development fluorescent signals were collected at each of these intervals. The samples were subjected to LATE-PCR amplification for 70 cycles of 95° C. at 10 sec, 66° C. at 20 sec., and 72° C. at 20 sec. After PCR amplification the samples were cooled to 30° C. at 0.1° C. per sec to allow formation of probe-target hybrids. The samples were heated up back to 72° C. at 1° C. intervals for 30 seconds each at a rate of 2° C.-3° C./sec. For the purpose of assay development fluorescent signals were once against collected at each of these intervals.

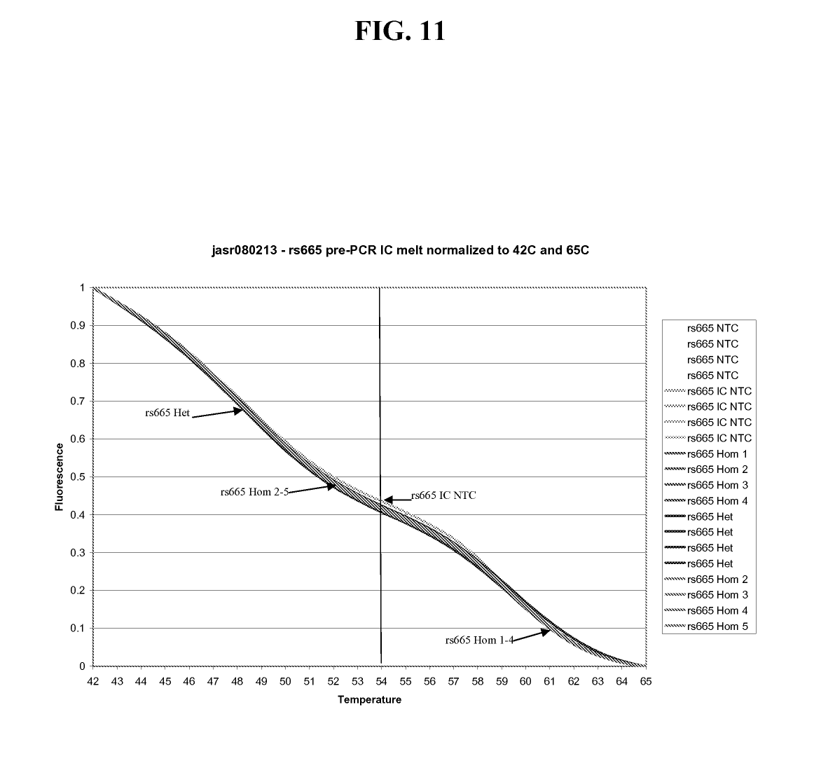

For pre-PCR fluorescent signal analysis, the first derivative of the melting curve was calculated to identify the three temperatures needed for fluorescent signal normalization, as discussed above (see FIG. 11).

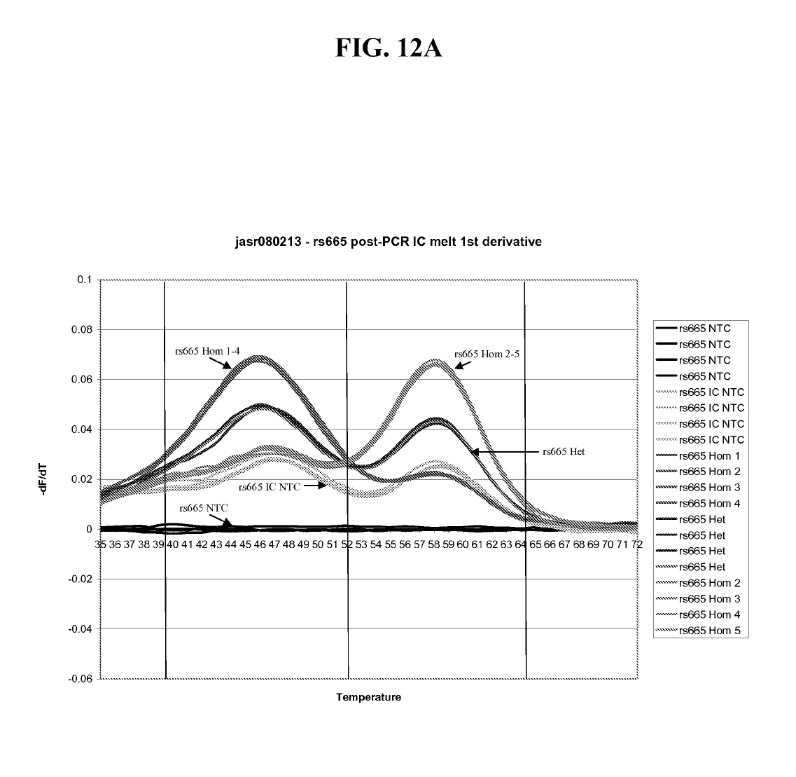

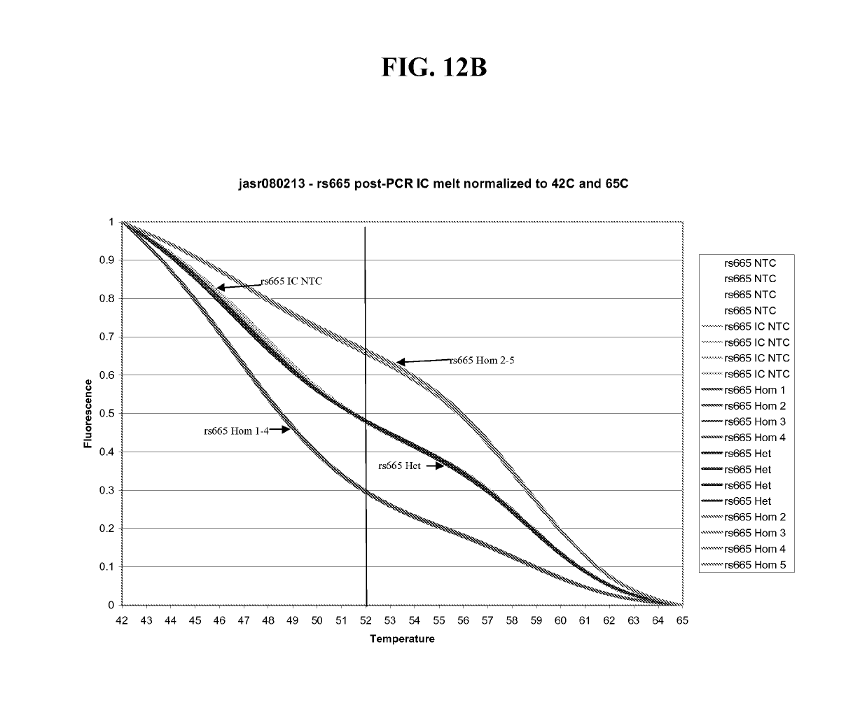

The raw fluorescent signals were normalized at the upper and the lower temperatures to determine the pre-PCR fluorescent signal ratios for the non-amplifiable control at all temperatures (FIGS. 12A-B). Following PCR the same 1^{st} derivative and fluorescent signal ratio analysis was performed (FIG. 13). The pre-PCR fluorescent signals were then subtracted from the post-PCR fluorescent signals to obtain the adjusted post-PCR fluorescent signals derived exclusively from the PCR products. The adjusted post-PCR fluorescent signals were then used to determine the post-PCR fluorescent signal ratio from the amplified PCR products. The resulting PCR products fluorescent ratio was then compared to the pre-PCR non-amplifiable control fluorescence ratios by subtraction (FIGS. 14A-C). The results clearly show that the genotypes can be clearly identified on a per sample basis using the internal control as a reference (FIGS. 14A-C).

All publications and patents mentioned in the present application are herein incorporated by reference. Various modification and variation of the described methods and compositions will be apparent to those skilled in the art without departing from the scope and spirit of the assays described herein. Although the methods, compositions, and kits have been described in connection with specific exemplary embodiments, it should be understood that the claims should not be unduly limited to such specific embodiments. Indeed, various modifications of the described modes for carrying out the assays described herein that are obvious to those skilled in the relevant fields are intended to be within the scope of the following claims.

1-27. (canceled)

28. A method for preparing a mismatch tolerant probe, comprising:
a) selecting a candidate probe sequence that is perfectly complementary to one allele of a variable DNA target sequence, or, single nucleotide polymorphic DNA target sequence, said probe sequence having 5′ and 3′ ends; and
b) designing a probe from said candidate probe sequence by introducing one or more of the following modifications to said candidate probe sequence: i) addition of a nucleotide tail at said 5′ and/or 3′ ends of said candidate probe sequence of at least 2 nucleotides that are not complementary to the target sequence; ii) addition of a mismatch nucleotide at least one nucleotide away from the position of the polymorphic allele on said target sequence; and iii) positioning the portion of said candidate probe sequence that hybridizes to the polymorphic target sequence at least 2 nucleotides away from either the 5′ or 3′ ends of said probe.

29. The method of claim 28 where said probe is covalently labeled with one or more moieties selected from the group consisting of a fluorophore and a quencher.

30. The method of claim 28, wherein said probe hybridizes to at least 90% of the DNA
target complementary at the site of the variable sequence and less than 10% of the DNA target mismatched at the site of the variable sequence at a first hybridization temperature.

31. The method of claim 28, wherein said probe hybridizes to at least 90% of all allelic variants at the polymorphic site at a second hybridization temperature.

32. The method of claim 28, wherein said DNA target is an amplified single strand.

33. A probe produced by the method of claim 28.

34. An Assay constructed by mixing at least one mismatch tolerant probe prepared by the method of claim 28 and a sample suspected of containing said variable DNA target sequence, or, single nucleotide polymorphic DNA target sequence and detecting a probe/target hybridization complex in said mixture.

35. The Assay of claim 34, further comprising:
a) adding to said mixture primers, enzyme, and all other reagents required for asymmetric amplification of said target sequence;
b) then adding to said mixture a non-amplifiable oligonucleotide control target sequence comprising a binding site for said at least one mismatch-tolerant probe;
c) then contacting said non-amplifiable control target sequence and said at least one mismatch tolerant probe and measuring a pre-amplification fluorescence signal at one or more temperatures resulting from hybridization of said control target sequence and said probe;
c) then performing asymmetric amplification to generate an amplified single strand variable DNA target sequence, or, single nucleotide polymorphic DNA target sequence;
d) then measuring the measuring a post-amplification fluorescent signal at one or more temperatures resulting from hybridization of said probe to at least one target sequence;
e) then subtracting said pre-amplification fluorescence signal from said post-amplification fluorescent signal to obtain an adjusted post-amplification fluorescent signal; and
f) then normalizing differences in the adjusted post-PCR fluorescent signals among replicate samples by dividing each said adjusted post-amplification fluorescent signal by its corresponding said pre-amplification fluorescent signal.

36. A method of asymmetric PCR amplification, comprising:
a) contacting a mismatch tolerant probe with a non-amplifiable control sequence added prior to the start of the amplification reaction, wherein said non-amplifiable internal control sequences comprises: i) a first 3′ blocked oligonucleotide that is not complementary to the primers used in said asymmetric PCR and contains a first binding site complementary to a mismatch-tolerant nucleic acid probe, said sequence including a first allelic variant of a variable DNA target sequence, or, single nucleotide polymorphic DNA target sequence; ii) a second 3′ blocked oligonucleotide target that is not complementary to the primers used in said asymmetric PCR comprising and contains a second binding site complementary to said mismatch-tolerant nucleic acid probe, said sequence including a second allelic variant of said variable DNA target sequence, or, single nucleotide polymorphic DNA target sequence;
b) measuring fluorescence signal prior to PCR amplification at three temperatures comprising a high temperature where said mismatch-tolerant probe does not bind to either of said oligonucleotides, a middle temperature wherein said mismatch tolerant probe binds to at least 90% of said first oligonucleotide target and to at least 10% or less of the second oligonucleotide target, and a third temperature where said mismatch-tolerant probe binds to at least 90% of both of said first and second oligonucleotides;
c) calculating a three-temperature pre-PCR fluorescent ratio, wherein said fluorescent ratio defines a reference fluorescent ratio for the molar ratio of said first and second oligonucleotides;
d) performing an asymmetric PCR reaction to detect said variable DNA target sequence, or, single nucleotide polymorphic DNA target sequence in a sample;
e) repeating step b) to obtain post-PCR fluorescence signals at each of the three said temperatures;
f) subtracting said pre-PCR reference fluorescence signals from said post-PCR fluorescent signals to obtain adjusted post-PCR fluorescent signals;
g) repeating said steps c) using the adjusted post-PCR fluorescent signals to obtain post-PCR three-temperature fluorescence signal ratios; and
h) subtracting said reference pre-PCR fluorescence signal ratio values from said post-PCR fluorescence signal ratio values to generate an adjusted fluorescence signal ratio according to the formula (Fs-Ft)/(Fb-Ft), where Ft is the fluorescence at said first detection temperature, Fb is the fluorescence at said low temperature, and Fs is the fluorescence at said intermediate temperature.

37. The method of claim 36, wherein said first and second oligonucleotide control target sequences are at a predetermined molar ratio.

38. The method of claim 36, wherein similar pre-PCR reference fluorescence signal ratios and adjusted fluorescence signal ratios are indicative the ratio at which allelic variants of a variable DNA target sequence, or, single nucleotide polymorphic DNA target sequence are present in a sample.

39. The method of claim 36, wherein different pre-PCR reference fluorescence signal ratios and adjusted fluorescence signal ratios are indicative of a DNA sample where said polymorphic alleles targets are at different molar ratios as said first and second oligonucleotides.

40. The method of claim 36, wherein said first and second oligonucleotide targets are at equimolar ratio.

41. The method of claim 40, wherein similar pre-PCR reference fluorescence signal ratios and adjusted fluorescence signal ratios are indicative of a DNA sample heterozygous for variants in a variable DNA target sequence, or, single nucleotide polymorphic DNA target sequence.

42. The method of claim 40, wherein pre-PCR reference fluorescence signal ratios different than adjusted fluorescence signal ratios are indicative of a DNA sample not heterozygous for said variable DNA target sequence, or, single nucleotide polymorphic DNA target sequence.

43. The method of claim 40, wherein difference pre-PCR reference fluorescence signal ratios and adjusted fluorescent signal ratios detected in sample from an individual known to be heterozygous for the tested allelic variants indicative of allelic imbalances due to copy number variations at said variable site.

44. The method of claim 36, wherein said oligonucleotides comprise at least 6 nucleotides flanking each side of said first and second binding site for said mismatched-tolerant probe

45. A method for identifying the presence of variants in a variable DNA target sequence, or, single nucleotide polymorphic DNA target sequence in a sample, comprising:
a) providing: i) a sample suspected of containing: a nucleic acid sequence comprising a first and/or second variant of a variable DNA target sequence, or, single nucleotide polymorphic DNA target sequence, ii) a labeled probe, wherein said probe comprises one or more mismatches to said nucleic acid sequence at a location distinct from the variable DNA target sequence, or, single nucleotide polymorphic DNA target sequence, iii) a first variant temperature signal ratio, iv) a second variant temperature signal ratio, v) a temperature signal ratio indicative of the presence of both the first and the second variants, vi) a forward primer, and vii) a reverse primer;
b) combining said sample, said labeled probe, said forward primer, and said reverse primer to generate a combined sample and treating said combined sample under asymmetric amplification conditions such that: a first single-stranded amplicon is generated if said first variant is present, and a second single-stranded amplicon is generated if said second variant is present, wherein said first and second single-stranded amplicons each comprise the following identical sequences:
i) a 5′ end corresponding to the sequence of said reverse primer, and
ii) a 3′ end complementary to said forward primer;
and wherein said first and second single-stranded amplicons do not have complete sequence identity;
c) exposing said combined sample to multiple temperatures that allow said labeled probe to hybridize to said probe hybridization sequence and produce temperature-dependent signals;
d) detecting said temperature-dependent signals at at least two temperatures;
e) generating an experimental temperature-dependent signal ratio; and
f) comparing said experimental temperature-dependent signal ratio with said first, second and first/second variant temperature-dependent signal ratios, wherein a match between said experimental temperature-dependent signal ratio and said first or second or first/second variant temperature-dependent signal ratio identifies the presence of said first and/or second variant in said sample.

46. The method of claim 45, wherein said asymmetric PCR is LATE-PCR.

47. The method of claim 45, wherein said labeled probe comprises with one of the following modifications: i) addition of a nucleotide tail at said 5′ and/or 3′ ends of said probe sequence of at least 2 nucleotides that are not complementary to said polymorphic target sequence; ii) addition of a mismatch nucleotide at least one nucleotide away from the position of said polymorphic target sequence; and iii) positioning the portion of the probe sequence that hybridizes to said polymorphic target sequence at least 2 nucleotides away from either the 5′ or the 3′ end of said nucleic acid sequence.


SEQUENCE LISTING (20 sequences)

SEQ ID NO: 1 - DNA, 32 bp, Artificial Sequence
  Synthetic
ccgtgcctggccaacacagtatttaaaaacaa

SEQ ID NO: 2 - DNA, 25 bp, Artificial Sequence
  Synthetic
gtagagtacagtgctaagccatatt

SEQ ID NO: 3 - DNA, 17 bp, Artificial Sequence
  Synthetic
taccttaggctccaata

SEQ ID NO: 4 - DNA, 26 bp, Artificial Sequence
  Synthetic
gcccagccggtgtcattttctgatcc

SEQ ID NO: 5 - DNA, 20 bp, Artificial Sequence
  Synthetic
caatcccttgacctgttgtg

SEQ ID NO: 6 - DNA, 20 bp, Artificial Sequence
  Synthetic
ctcttcagctcgaacaatag

SEQ ID NO: 7 - DNA, 27 bp, Artificial Sequence
  Synthetic
ggaggtcagagtacccactgctccttc

SEQ ID NO: 8 - DNA, 22 bp, Artificial Sequence
  Synthetic
gctcctgagcaatgagaatgtc

SEQ ID NO: 9 - DNA, 18 bp, Artificial Sequence
  Synthetic
cttgcaggtgggatagga

SEQ ID NO: 10 - DNA, 30 bp, Artificial Sequence
  Synthetic
gcatgctcctatcccacctgcaaggggttg

SEQ ID NO: 11 - DNA, 30 bp, Artificial Sequence
  Synthetic
gcatgctcctatcccacctacaaggggttg

SEQ ID NO: 12 - DNA, 25 bp, Artificial Sequence
  Synthetic
ttcaaattcgtccgtatgtactcct

SEQ ID NO: 13 - DNA, 19 bp, Artificial Sequence
  Synthetic
atagtacatacggacgaat

SEQ ID NO: 14 - DNA, 25 bp, Artificial Sequence
  Synthetic
ttcaaattcgtccatatgtactcct

SEQ ID NO: 15 - DNA, 26 bp, Artificial Sequence
  Synthetic
ctcaatcatgacggaagatgaaggag

SEQ ID NO: 16 - DNA, 14 bp, Artificial Sequence
  Synthetic
catctttcgtcatg

SEQ ID NO: 17 - DNA, 26 bp, Artificial Sequence
  Synthetic
ctcaatcatgacagaagatgaaggag

SEQ ID NO: 18 - DNA, 35 bp, Artificial Sequence
  Synthetic
tggtgtatgttggaataaatatcgaatataaattt

SEQ ID NO: 19 - DNA, 28 bp, Artificial Sequence
  Synthetic
tttatattcgatatttattccaacatac

SEQ ID NO: 20 - DNA, 35 bp, Artificial Sequence
  Synthetic
tggtgtatgttggaataaatatcgaatacaaattt